(19) United States
(12) Patent Application Publication
(10) Pub. No.: US 2021/0228061 A1
(43) Pub. Date: Jul. 29, 2021

(54) MEDICAL OBSERVATION SYSTEM, MEDICAL OBSERVATION APPARATUS, AND DRIVE METHOD OF MEDICAL OBSERVATION APPARATUS

(71) Applicant: Sony Corporation, Tokyo (JP)

(72) Inventors: Takeshi MATSUI, Tokyo (JP); Tetsuro KUWAYAMA, Tokyo (JP); Goro FUJITA, Tokyo (JP); Hiroshi YOSHIDA, Tokyo (JP); Takanori FUKAZAWA, Tokyo (JP); Fumisada MAEDA, Tokyo (JP)

(73) Assignee: Sony Corporation, Tokyo (JP)

(21) Appl. No.: 17/257,022

(22) PCT Filed: Jul. 2, 2019
(86) PCT No.: PCT/JP2019/026374

§ 371 Date: Dec. 30, 2020

(30) Foreign Application Priority Data
Jul. 6, 2018 (JP) 2018-129186

(51) Int. Cl. A61B 1/00; A61B 1/313; A61B 1/018

(52) U.S. Cl. CPC A61B 1/00006; A61B 1/3137; A61B 1/00009; A61B 1/018; A61B 1/00043

(57) ABSTRACT

A medical observation system (3) including: an imaging portion (303) configured to capture an image of an affected area; a detecting portion (311) configured to extract; a movement of a treatment tool held in a vicinity of the affected area based on images of the affected area having been sequentially captured by the imaging portion and to detect a movement of the affected area based on a result of the extraction; and a control portion (301) configured to control processing related to observation of the affected area in accordance with a detection result of the movement of the affected area.

### TECHNICAL FIELD

The present disclosure relates to a medical observation system, a medical observation apparatus, and a drive method of a medical observation apparatus.

### BACKGROUND ART

In recent years, due to advances in surgical techniques and surgical instruments, surgeries (so-called microsurgeries) for carrying out various kinds of treatment while observing an affected area with a medical observation apparatus such as a surgical microscope or an endoscope are being frequently performed. In addition, such medical observation apparatuses are not limited to apparatuses that enable an affected area to be optically observed, and apparatuses are being proposed that cause an image of an affected area having been captured by an imaging apparatus (a camera) or the like to be displayed as an electronic image on a display apparatus such as a monitor. Such developments in technology enable a large number of objects to be observed. For example, PTL 1 discloses an example of a technique that enables a blood flow to be observed.

### CITATION LIST

### Patent Literature

[PTL 1]
JP 2017-170064A

### SUMMARY

### Technical Problem

In situations where a procedure is performed while observing an affected area with a medical observation apparatus, pulsation or the like may cause the affected area to be an observation object to exhibit a movement such as vibration regardless of the presence or absence of an intervening procedure. As a specific example, in a case where a blood vessel or a vicinity thereof is to be an observation object such as the observation of an aneurysm, a situation where an affected area vibrates due to pulsation can be envisaged. In such a situation, for example, the movement exhibited by the affected area such as vibration may make it difficult to accurately observe the affected area. As a specific example, in a case where flow of blood is observed after clipping an aneurysm, vibration of the aneurysm caused by pulsation or the like may make it difficult to accurately observe the aneurysm.

In consideration thereof, the present disclosure proposes a technique that, enables observation of an affected area to be realized in a more preferable mode even in a situation where the affected area can move regardless of the presence or absence of an intervening procedure.

### Solution to Problem

The present disclosure provides a medical observation system including: an imaging portion configured to capture an image of an affected area; a detecting portion configured to extract a movement of a treatment tool held in a vicinity of the affected area based on images of the affected area having been sequentially captured by the imaging portion and to detect a movement of the affected area based on a result of the extraction; and a control portion configured to control processing related to observation of the affected area in accordance with a detection result of the movement of the affected area.

In addition, the present disclosure provides a medical observation apparatus including: a detecting portion configured to extract a movement of a treatment tool held in a vicinity of an affected area based on images of the affected area having been sequentially captured by an imaging portion and to detect a movement of the affected area based on a result of the extraction; and a control portion configured to control processing related to observation of the affected area in accordance with a detection result of the movement of the affected area.

Furthermore, the present disclosure provides a drive method of a medical observation apparatus including the steps performed by a computer of: extracting a movement of a treatment tool held in a vicinity of an affected area based on images of the affected area having been sequentially captured by an imaging portion and detecting a movement of the affected area based on a result of the extraction; and controlling processing related to observation of the affected area in accordance with a detection result of the movement of the affected area.

### Advantageous Effects of Invention

As described above, according to the present disclosure, a technique is provided that enables observation of an affected area to be realized in a more preferable mode even in a situation where the affected area can move regardless of the presence or absence of an intervening procedure.

It should be noted that the advantageous effect described above is not necessarily restrictive and, in addition to the advantageous effect described above or in place of the advantageous effect described above, any of the advantageous effects described in the present specification or other advantageous effects that can be comprehended from the present specification may be produced.

### BRIEF DESCRIPTION OF DRAWINGS

FIG. 1 is a diagram showing an example of a configuration of a system including a surgical video microscope apparatus to which the technique according to the present disclosure can be applied.

FIG. 2 is an explanatory diagram for explaining an example of a procedure.

FIG. 3 is an explanatory diagram for explaining an example of a situation where an affected area moves accompanying pulsation.

FIG. 4 is an explanatory diagram for explaining an example of a configuration of a medical observation system according to an embodiment of the present disclosure.

FIG. 5 is an explanatory diagram for explaining a fundamental concept of technical features of the medical observation system according to the embodiment.

FIG. 6 is a block diagram showing an example of a functional configuration of the medical observation system according to the embodiment.

FIG. 7 is a flow chart showing an example of a flow of a series of processing by the medical observation system according to the embodiment.

FIG. 8 is an explanatory diagram for explaining an example of control according to Example 1.

FIG. 9 is an explanatory diagram for explaining an example of control according to Example 2.

FIG. 10 is an explanatory diagram for explaining another example of control according to Example 2.

FIG. 11 is an explanatory diagram for explaining an example of control according to Example 3.

FIG. 12 is a functional block diagram showing a configuration example of a hardware configuration of an information processing apparatus that constitutes a medical observation system according to an embodiment of the present disclosure.

FIG. 13 is a diagram showing an example of a schematic configuration of an endoscopic surgery system according to an application of an embodiment of the present disclosure.

FIG. 14 is a block diagram showing an example of functional configurations of a camera head and a CCU shown in FIG. 13.

### DESCRIPTION OF EMBODIMENTS

Hereinafter, a preferred embodiment of the present disclosure will be described in detail with reference to the accompanying drawings. In the present specification and the drawings, components substantially having a same functional configuration will be denoted by same reference signs and overlapping descriptions thereof will be omitted.

Descriptions will be given in the following order.

1. Configuration example of medical observation system

2. Examination of observation using medical observation system

3. Technical features

3.1 Configuration example of system

3.2 Fundamental concept

3.3 Functional configuration

3.4 Processing

3.5 Examples

4. Example of hardware configuration

5. Applications

6. Conclusion

### 1. CONFIGURATION EXAMPLE OF MEDICAL OBSERVATION SYSTEM

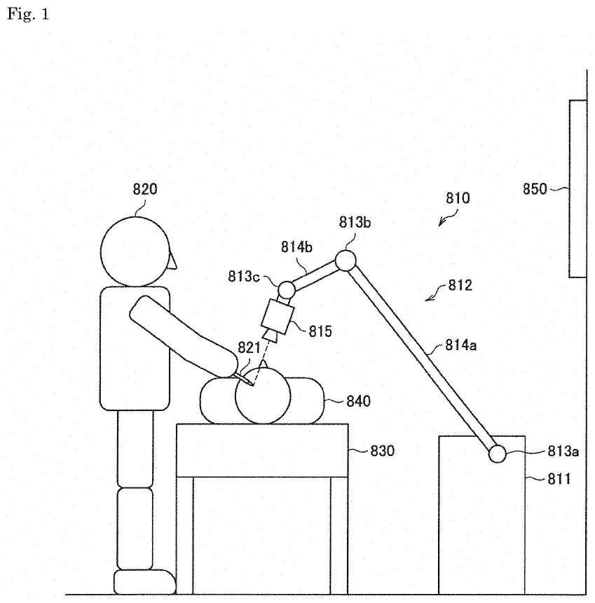

First, as an example of a schematic configuration of a medical observation system to which the technique according to an embodiment of the present disclosure can be applied, an example of a system including a so-called surgical video microscope apparatus will be described with reference to FIG. 1.

For example, FIG. 1 is a diagram which shows an example of a configuration of a system including a surgical video microscope apparatus to which the technique according to the present disclosure can be applied and which schematically represents how a procedure using the surgical video microscope apparatus is performed. Specifically, FIG. 1 shows how a medical doctor who is a practitioner (user) 820 uses a surgical instrument 821 such as a scalpel, tweezers, or forceps to perform surgery on a subject to be operated upon (a patient) 840 on an operating table 830. It should be noted that, in the following description, a procedure is used as a collective term for various kinds of medical treatment such as a surgery and a test to be performed by a medical doctor who is the user 820 on a patient who is the subject to be operated upon 840. In addition, while the example shown in FIG. 1 illustrates how a surgery as an example of a procedure is performed, the procedure in which a surgical video microscope apparatus 810 is used is not limited to a surgery and may be a procedure of various other kinds.

The surgical video microscope apparatus 810 is provided beside the operating table 830. The surgical video microscope apparatus 810 includes a base portion 811 that is a base, an arm portion 812 that extends from the base portion 811, and an imaging unit 815 which is connected as a tip unit to a tip of the arm portion 812. The arm portion 812 has a plurality of joint portions 813a, 813b, and 813c, a plurality of links 814a and 814b that are coupled by the joint portions 813a and 813b, and the imaging unit 815 that is provided at the tip of the arm portion 812. While the arm portion 812 has three joint portions 813a to 813c and two links 814a and 814b in the example shown in FIG. 1 for the sake of simplicity, in reality, the numbers and shapes of the joint portions 813a to 813c and the links 814a and 814b, directions of drive axes of the joint portions 813a to 813c, and the like may be appropriately set so as to realize a desired degree of freedom in consideration of positions and degrees of freedom of postures of the arm portion 812 and the imaging unit 815.

The joint portions 813a to 813c have a function of rotatably coupling the links 814a and 814b to each other and, by driving rotations of the joint portions 813a to 813c, drive of the arm portion 812 is controlled. In the following description, a position of each component of the surgical video microscope apparatus 810 refers to a position (coordinates) in a space defined for drive control, and a posture of each component refers to an orientation (an angle) relative to an arbitrary axis in the space defined for drive control. In addition, in the following description, driving (or performing drive control) of the arm portion 812 refers to driving (or performing drive control) of the joint portions 813a to 813c and changing (or controlling a change in) a position and a posture of each component of the arm portion 812 by driving (or performing drive control) of the joint portions 813a to 813c.

The imaging unit 815 is connected as a tip unit to the tip of the arm portion 812. The imaging unit 815 is a unit that acquires an image of an imaging object and is, for example, a camera or the like capable of capturing a moving image or a still image. As shown in FIG. 1, the postures and positions of the arm portion 812 and the imaging unit 815 are controlled by the surgical video microscope apparatus 810 so that the imaging unit 815 provided at the tip of the arm portion 812 images a situation of a procedure site of the subject to be operated upon 840. A configuration of the imaging unit 815 that is connected as a tip unit to the tip of the arm portion 812 is not particularly limited and, for example, the imaging unit 815 is configured as a microscope that acquires an enlarged image of an imaging object. In addition, the imaging unit 815 may be configured to be attachable to and detachable from the arm portion 812. According to such configurations, for example, the imaging unit 815 in accordance with usage application may be appropriately connected as a tip unit to the tip of the arm portion 812. For example, an imaging apparatus to which is applied a branching optical system according to the embodiment described earlier can be applied as the imaging unit 815. In other words, in the present application, the imaging unit 815 or the surgical video microscope apparatus 810 including the imaging unit 815 can correspond to an example of the “medical observation apparatus”. In addition, while the present description focuses on a case where the imaging unit 815 is applied as the tip unit, the tip unit to be connected to the tip of the arm portion 812 is not necessarily limited to the imaging unit 815.

In addition, a display apparatus 850 such as a monitor or a display is installed at a position that opposes the user 820. An image of the procedure site having been captured by the imaging unit 815 is displayed as an electronic image on a display screen of the display apparatus 850. The user 820 performs various kinds of treatment while viewing the electronic image of the procedure site that is displayed on the display screen of the display apparatus 850.

According to the configuration described above, a surgery can be performed while imaging a procedure site with the surgical video microscope apparatus 810.

While an example of a surgical video microscope apparatus has been described above, a portion corresponding to the surgical video microscope apparatus may be configured as a so-called optical microscope apparatus in this case, an optical microscope unit need only be connected as the tip unit to be connected to the tip of the arm portion 812. In addition, the microscope unit may include an imaging apparatus.

This concludes the description of an example of a case where the medical observation system according to an embodiment of the present disclosure is configured as a microscope imaging system including a microscope unit with reference to FIG. 1 as an example of a schematic configuration of the medical observation system.

### 2. EXAMINATION OF OBSERVATION USING MEDICAL OBSERVATION SYSTEM

Next, when a situation is assumed where an affected area (for example, an observation object such as an aneurysm) inside the body of a patient is observed using the medical observation system, technical problems that may arise when performing the observation will be described below using specific examples.

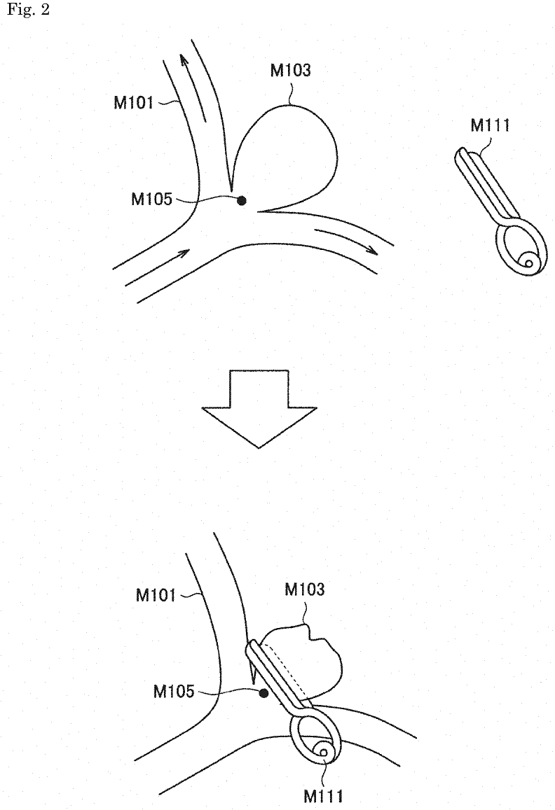

For example, FIG. 2 is an explanatory diagram for explaining an example of a procedure and shows an outline of an example of an unruptured cerebral aneurysm clipping surgery In an unruptured cerebral aneurysm clipping surgery, in order to prevent a disorder due to a rupture of an aneurysm (for example, a cerebral aneurysm) from occurring, a part of a blood vessel is clipped using a titanium clip or the like to suppress flow of blood into the aneurysm (in other words, the aneurysm is occluded). For example, the example shown in FIG. 2 represents a case where an aneurysm occurring in a part of a blood vessel M101 is occluded by clipping using a dip M111. In FIG. 2, an upper diagram represents a state prior to the clipping and a lower diagram represents a state during the clipping. In addition, in FIG. 2, a reference sign M103 denotes a dome of the aneurysm. Furthermore, a reference sign M105 denotes a neck of the aneurysm. In other words, in the example shown in FIG. 2, blood flowing through the blood vessel M101 is prevented from flowing into the aneurysm by applying the clip M111 to the neck M105 of the aneurysm.

In the clipping surgery described above, measures for preventing a rupture of an aneurysm may be taken after clipping by collapsing the aneurysm by a puncture or the like and causing blood to clot to form a thrombus. In order to prevent a rupture of an aneurysm by causing blood to clot to form a thrombus, for example, it is important to suppress inflow of blood into the aneurysm or minimize blood flow so that a blood flow rate drops to a level that causes blood to clot and induces the formation of a thrombus.

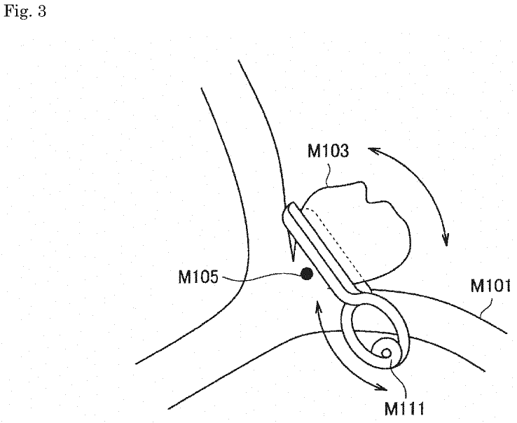

On the other hand, in a situation where the clipping surgery described above is applied, pulsation may cause an affected area (for example, an aneurysm) of a blood vessel or the like to exhibit a movement such as vibration and make an accurate observation difficult. For example, FIG. 3 is an explanatory diagram for explaining an example of a situation where an affected area moves accompanying pulsation. The example shown in FIG. 3 represents an example of a case where an aneurysm is occluded by applying the clip M111 to the neck M105 of the aneurysm in a similar manner to the example shown in FIG. 2. In this case, the blood vessel M101 may vibrate due to pulsation and, for example, the vibration may become evident as a movement of the aneurysm (a movement of the dome M103) or a movement of the clip M111 being applied to the aneurysm.

In addition, in a situation where an aneurysm is vibrating due to pulsation or the like, the vibration may cause a state of a flow of blood into the aneurysm to change and result in an observation as though the aneurysm is not occluded (in other words, a false-positive observation being made) despite inflow of blood to the aneurysm being occluded by clipping. In such a situation, for example, measures such as additionally applying a clip may be taken which may increase time or cost. For example, an increase in duration of a procedure may possibly cause an increase in a burden on a patient. In addition, a situation where it is difficult to observe a state of an affected area due to vibration or the like may possibly cause an increase in a burden on a medical doctor.

In addition, in order to confirm occlusion of an aneurysm by the clipping surgery described above, for example, a method using a fluorescent substance such as ICG may be applied. Specifically, in the method, a fluorescent substance such as ICG is injected into blood by an intravenous injection and, by irradiating light of a wavelength at which the fluorescent substance is excited and spectrally detecting excitation light, the presence or absence of inflow of blood into the aneurysm is confirmed. When ICG is used as the fluorescent substance, the confirmation described above is performed by, for example, irradiating near-infrared light with a wavelength in the vicinity of 800 nm and spectrally detecting excitation light with a wavelength in the vicinity of 830 nm using a filter or the like.

On the other hand, once a fluorescent substance such as ICG is injected into blood, the fluorescent substance such as ICG requires time to be discharged (washed out). Therefore, for example, when occlusion of an aneurysm by a clip is not sufficient and blood flows into the aneurysm, even if the inflow of blood into the aneurysm is stopped by once again occluding the aneurysm with an additional clip or the like, since the fluorescent substance such as ICG having flowed into the aneurysm together with blood produces fluorescent light, it may be difficult to determine whether or not the reocclusion had been sufficient.

In addition, other examples of methods of observing blood flow include a method referred to as LSCI (Laser Speckle Contrast Imaging). In LSCI, the presence or absence of a flow of blood is detected by irradiating dispersed material such as red blood cells in the blood with laser light and observing dispersed light. Due to such characteristics, even in LSCI, it may be difficult to observe an affected area due to movement of the affected area caused by pulsation or the like. In addition, in LSCI, since it is difficult to obtain a visible light image in a situation where an image is acquired by irradiating near-infrared light, for example, even if a clip is applied to an affected area, it may be difficult to confirm a position of the clip.

In consideration of the situations described above, the present disclosure proposes a technique that enables observation of an affected area to be realized in a more preferable mode even in a situation where, for example, the affected area can move due to an effect of pulsation or the like regardless of the presence or absence of an intervening procedure.

### 3. TECHNICAL FEATURES

Hereinafter, technical features of the medical observation system according to the embodiment of the present disclosure will be described.

### 3.1 CONFIGURATION EXAMPLE OF SYSTEM

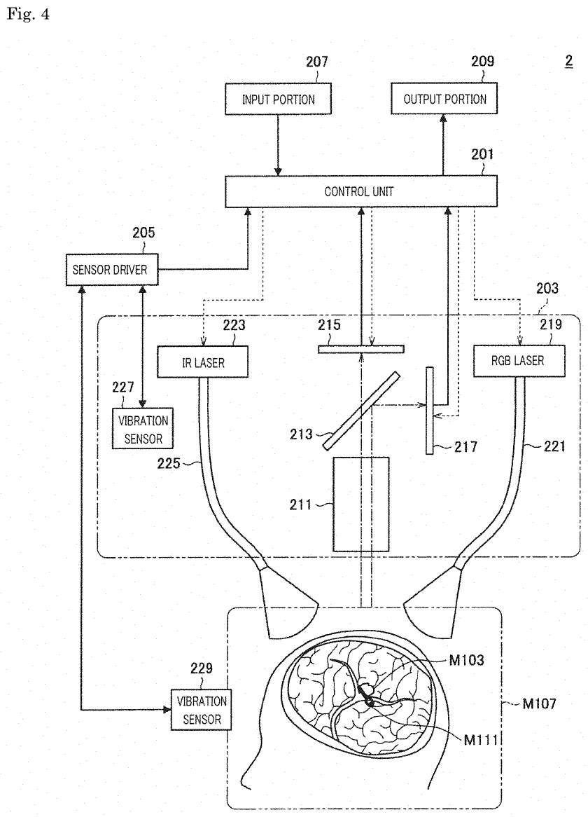

First, an example of a configuration of the medical observation system according to the embodiment of the present disclosure will be described. For example, FIG. 4 is an explanatory diagram for explaining an example of a configuration of the medical Observation system according to the embodiment of the present disclosure and shows an example of a system configuration when applying an ISM. In other words, the example shown in FIG. 4 has a configuration that assumes a situation where an affected area (an aneurysm) is observed by irradiating infrared light or visible light when the clipping surgery described above is applied on the aneurysm as an observation object. It should be noted that, in the following description, the medical observation system shown in FIG. 4 is also referred to as a “medical observation system 2” for convenience's sake.

In the example shown in FIG. 4, the medical observation system 2 includes a control unit 201, an imaging unit 203, a sensor driver 205, an input portion 207, and an output portion 209. The input portion 207 is an input interface with respect to the medical observation system 2. A user can input various kinds of information and various instructions to the medical observation system 2 via the input portion 7. In addition, the output portion 209 corresponds to the display apparatus 850 in the example shown in FIG. 1.

For example, the imaging unit 203 includes an imaging optical system 211, a branching optical system 213, imaging elements 215 and 217, an RGB laser 219, an IR (for example, near-infrared beam) laser 223, and a vibration sensor 227.

Each of the RGB laser 219 and the IR laser 223 corresponds to a light source apparatus for irradiating an affected area with light of a predetermined wavelength.

The RGB laser 219 is a light source that irradiates visible light and, for example, while the RGB laser 219 is constituted by respective lasers of red (wavelength of around 650 nm), green (wavelength of around 530 nm), and blue (wavelength of around 450 nm), the RGB laser 219 may alternatively be an LED light source and may be configured to produce white by exciting a fluorescent substance with a laser and an LED or with a laser. This configuration is utilized as a light source when, for example, a bright field image of an affected area is acquired. Specifically, visible light emitted from the RGB laser 219 is transmitted via a transmission cable 221 configured to be capable of guiding light using optical fibers and the like and irradiates the affected area. Accordingly, a bright field image of the affected area is condensed by the imaging optical system 211 to be described later.

The IR laser 223 is a light source that irradiates infrared light (IR light) and is used as a light source when, for example, fluorescent observation is performed. Specifically, infrared light emitted from the IR laser 223 is transmitted via a transmission cable 225 configured to be capable of guiding light using optical fibers and the like and irradiates the affected area. Accordingly a fluorescent substance such as ICG having been injected into blood or the like is excited by the irradiation of the infrared light, and excitation light emitted from the fluorescent substance is condensed by the imaging optical system 211 to be described later.

The imaging optical system 211 schematically represents an optical system for acquiring an image of an affected area of an observation object. For example, the imaging optical system 211 can correspond to an endoscope or a microscope. The imaging optical system 211 focuses, via the branching optical system 213 to be described later, incident light on any of the imaging elements 215 and 217 that are positioned in a subsequent stage. Accordingly an image of the affected area of the observation object is to be captured by the imaging elements 215 and 217. The imaging optical system 211 may be configured to include a plurality of optical systems such as lenses.

The branching optical system 213 separates light of a part of wavelength bands from light of other wavelength bands among the incident light and focuses each beam of separated light on a different imaging element between the imaging elements 215 and 217. As a specific example, the branching optical system 213 is configured to include a dichroic filter or the like and separates light of a part of wavelength bands from light of other wavelength bands among the incident light by transmitting the light of a part of wavelength bands and reflecting the light of other wavelength bands. For example, in the example shown in FIG. 4, light transmitted through the branching optical system 213 is guided to the imaging element 215 and light reflected by the branching optical system 213 is guided to the imaging element 217. In the following description, for convenience's sake, it is assumed that light belonging to a visible light wavelength region is guided to the imaging element 215 and light with a longer wavelength than visible light (for example, light belonging to a near infrared wavelength region) such as infrared light and fluorescent light emitted by ICG is guided to the imaging element 217. However, the configuration of the branching optical system 213 is not necessarily limited to the example described above as long as incident light can be separated into a plurality of beams. In other words, the configuration of the branching optical system 213 may be modified as deemed appropriate according to a wavelength and an observation method of light to be an observation object, the configuration of the imaging unit 203, and the like.

The imaging element 215 is an imaging element which is provided in a stage subsequent to the branching optical system 213 and on which light belonging to the visible light wavelength region having been separated by the branching optical system 213 focuses. As the imaging element 215, for example, an imaging element such as a CCD or a CMOS with an RGB color filter can be applied.

The imaging element 217 is an imaging element which is provided in a stage subsequent to the branching optical system 213 and on which light with a longer wavelength than the visible light separated by the branching optical system 213 (for example, light belonging to the near infrared wavelength region) focuses. As the imaging element 217, an imaging element with higher sensitivity may be applied. As a specific example, an imaging element 217 such as a CCD or a CMOS not provided with a color filter or the like can be applied.

The vibration sensor 227 is a sensor that detects a movement (for example, vibration) of the imaging unit 203. For example, the vibration sensor 227 may include an acceleration sensor or an angular velocity sensor and detect a movement of a case (for example, an acceleration or an angular velocity that acts on the case) of the imaging unit 203. The vibration sensor 227 notifies the sensor driver 205 of a detection result of the movement of the imaging unit 203.

A vibration sensor 229 is a sensor that detects a movement of a predetermined site M107 of a patient (in other words, a movement of the patient). As a specific example, when a cerebral aneurysm is an observation object, the vibration sensor 229 is installed on a part of the head or the like of the patient so that a movement of the head can be detected as a movement of the site M107. The vibration sensor 229 notifies the sensor driver 205 of a detection result of the movement of the predetermined site M107 of the patient.

The sensor driver 205 controls operations of the various sensors and acquires information in accordance with detection results of various states from the sensors. As a specific example, the sensor driver 205 controls operations of the vibration sensor 227 and acquires information in accordance with a detection result of a movement (for example, vibration) of the imaging unit 203 from the vibration sensor 229. In addition, as another example, the sensor driver 205 controls operations of the vibration sensor 227 and acquires information in accordance with a detection result of a movement (for example, vibration) of the imaging unit 203 from the vibration sensor 229. The sensor driver 205 may execute control of operations of the various sensors and acquisition of information from the sensors based on control by the control unit 201. In addition, the sensor driver 205 may output information acquired from the various sensors to the control unit 201.

The control unit 201 may control operations of various light sources such as the RGB laser 219 and the IR, laser 223 in accordance with an observation object and an observation method. In addition, the control unit 201 may control operations related to capturing an image by at least any of the imaging elements 215 and 217. In this case, the control unit 201 may control imaging conditions (for example, a shutter speed, an aperture, and a gain) of an image. In addition, the control unit 201 may acquire an image in accordance with an imaging result by at least any of the imaging elements 215 and 217 and present the image to the output portion 209. Furthermore, in this case, the control unit 201 may perform predetermined image processing with respect to the acquired image. In addition, the control unit 201 may control operations of the respective portions in accordance with a detection result of various states. As a specific example, the control unit 201 may acquire information in accordance with a detection result of a movement of the imaging unit 203 by the vibration sensor 227 from the sensor driver 205 and execute so-called camera-shake compensation based on the information. In this case, the control unit 201 may compensate for a blur of the imaging unit 203 by cutting out a portion from an image according to the imaging results of the imaging elements 215 and 217 in accordance with the movement (in other words, the blur) of the imaging unit 203. In addition, the control unit 201 may execute the various kinds of processing described above in accordance with an instruction by the user which is input via the input portion 207.

It should be noted that the example described with reference to FIG. 4 is merely an example and does not necessarily limit a configuration of the medical observation system according to the embodiment of the present disclosure. In other words, a part of the configuration may be modified as deemed appropriate in accordance with an observation object and an observation method without deviating from a fundamental concept of the medical observation system according to the present embodiment to be described later.

This concludes the description of an example of the configuration of the medical observation system according to the embodiment of the present disclosure with reference to FIG. 4.

### 3.2 FUNDAMENTAL CONCEPT

Next, a fundamental concept of the technical features of the medical observation system according to the embodiment of the present disclosure will be described.

As described earlier, in a situation where the clipping surgery described above is applied, a movement such as vibration exhibited by an affected area (for example, an aneurysm) of a blood vessel or the like may make an accurate observation difficult. In consideration thereof; the medical observation system according to the present embodiment enables observation in a more preferable mode to be realized by detecting a movement of the affected area based on an image captured by an imaging unit or the like such as an endoscope apparatus or a microscope apparatus and executing various kinds of processing using a result of the detection. As a specific example, an image that enables a medical doctor to more readily make an accurate determination may be generated by controlling imaging conditions (for example, a shutter speed) of the imaging elements in accordance with a movement of an affected area such as an aneurysm. In addition, as another example, in a situation where a movement of the affected area is large, a warning can be issued to notify the medical doctor of a situation where making an accurate determination is difficult. An example of processing using a detection result of the movement of an affected area will be described in detail later.

In addition, the medical observation system according to the present embodiment uses a detection result of a movement of a treatment tool such as a clip which is held in a vicinity of an affected area to detect a movement of the affected area. Specifically, the medical observation system according to the present embodiment extracts a movement of a treatment tool such as a clip by extracting a characteristic portion of the treatment tool from images sequentially captured by the imaging unit or the like, and detects a movement of an affected area based on an extraction result of the movement of the treatment tool.

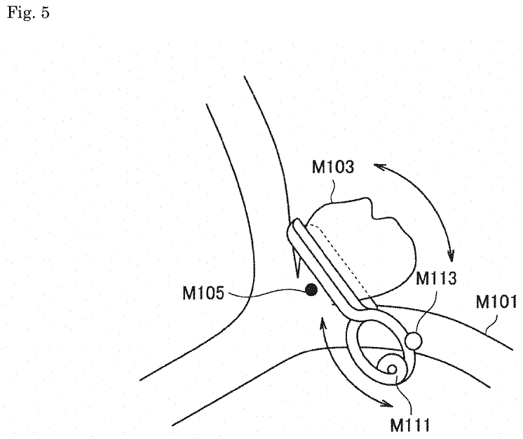

For example, FIG. 5 is an explanatory diagram for explaining a fundamental concept of technical features of the medical observation system according to the present embodiment and represents an example of a case where a movement of an affected area is detected by extracting a movement of a treatment tool from sequentially captured images. In the example shown in FIG. 5, blood flowing through the blood vessel M101 is prevented from flowing into the aneurysm by applying the clip M111 to the neck M105 of the aneurysm in a similar manner to the example described with reference to FIG. 2. In this case, in a similar manner to the example described with reference to FIG. 3, the blood vessel M101 vibrates due to pulsation and the vibration becomes evident as, for example, a movement of the aneurysm (a movement of the dome M103) or a movement of the clip M111 applied to the aneurysm. In this case, when the dome M103 of the aneurysm vibrates due to pulsation of the blood vessel M101 or the like, the clip M111 applied to the neck M105 of the aneurysm is to also vibrate as through in conjunction with the vibration of the aneurysm. In consideration thereof, the medical observation system according to the present embodiment extracts a movement of the clip M111 and detects a movement (for example, vibration) of the aneurysm using an extraction result of the movement of the clip M111.

As a specific example, in the example shown in FIG. 5, a light-emitting portion M113 is provided in a part of the clip M111, and the medical observation system according to the present embodiment extracts a movement of the clip M111 by extracting the light-emitting portion M113 from sequentially captured images. In addition, based on an extraction result of the movement of the clip M111, the medical observation system detects a movement of the dome M103 of the aneurysm (in other words, an affected area) to which the clip M111 is applied. Due to such a configuration, for example, even when it is difficult to directly view an affected area depending on an observation environment of the affected area or the observation method of the affected area, a movement of the affected area can be detected and processing in accordance with a result of the detection can be executed.

It should be noted that the example shown in FIG. 5 is merely an example and, as long as a movement of a treatment tool such as the clip M111 can be extracted and a movement of an affected area can be detected based on a result of the extraction of the movement, a configuration and method therefor are not particularly limited. For example, the extraction of a movement of a treatment tool to be an object is not limited to a method of extracting a light-emitting body such as that described above and a movement of the treatment tool can be extracted by extracting a portion having a predetermined feature such as a shape or a color among the treatment tool from sequentially captured images. As a specific example, by providing a portion in a color that differs from an observation object in at least a part of the treatment tool, a portion in the color in captured images can be extracted as a portion corresponding to the treatment tool. In addition, as another example, by providing a portion having a characteristic shape in at least a part of the treatment tool, a portion from which the shape has been extracted in the captured images can be extracted as a portion corresponding to the treatment tool.

In addition, a portion to become an index for extracting a treatment tool from an image such as the light-emitting body described above may be configured to be attachable to and detachable from the treatment tool. Adopting such a configuration enables, for example, only the portion to be an index to be detached. after completing a treatment such as clipping.

Furthermore, a configuration or a method for extracting a movement of a treatment tool can be modified as deemed appropriate in accordance with an envisaged observation environment or observation method. As a specific example, when fluorescent observation using a fluorescent substance that is excited by near-infrared light such as ICG is envisaged, a treatment tool such as a clip may be used in which at least a part thereof is constructed of a material which is excited by the near-infrared light and which emits light. In addition, as another example, a treatment tool such as a clip may be used in which at least a part thereof is coated with a paint which is excited by the near-infrared light and which emits light. Accordingly, even in a case where it is difficult to acquire a bright field image, a movement of a treatment tool can be extracted from acquired images and, based on a result of the extraction, a movement of an affected area where the treatment tool is held in a vicinity thereof can be detected.

In particular, in the field of medicine, an observation method may be selectively changed in accordance with an observation object and situations where an observation environment changes in accordance with the observation method can also be envisaged. Therefore, situations can be envisaged where, when observing a part of observation objects (for example, a blood flow), observation of another portion (for example, an aneurysm or a clipped position) becomes difficult and, as a result, accurate observation and diagnosis are inhibited. Even in such a case, with the medical observation system according to the embodiment of the present disclosure, modifying a configuration or a method for extracting a movement of a treatment tool as deemed appropriate in accordance with an observation environment or an observation method enables another portion in which the treatment tool is held in a vicinity of an observation object to be observed during an observation of the observation object.

This concludes the description of the fundamental concept of the technical features of the medical observation system according to the embodiment of the present disclosure with reference to FIG. 5.

### 3.3 FUNCTIONAL CONFIGURATION

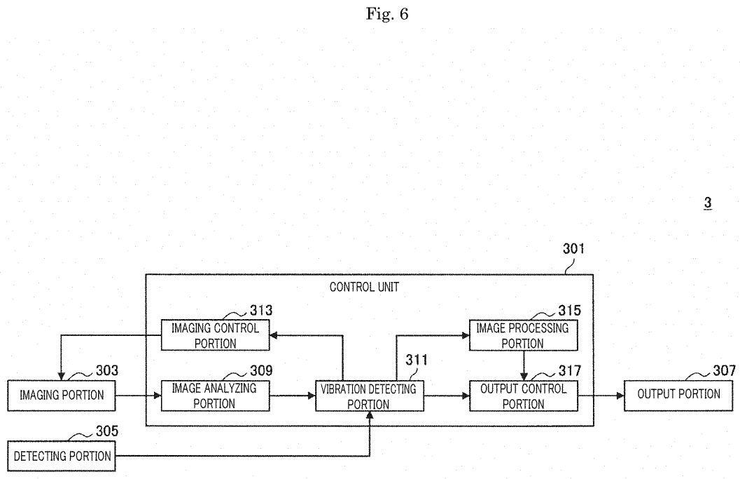

Next, an example of a functional configuration of the medical observation system according to the embodiment of the present disclosure will be described with a particular focus on an example of a functional configuration of a control unit that controls operations of various components of the medical observation system. For example, FIG. 6 is a block diagram showing an example of a functional configuration of the medical observation system according to the embodiment of the present disclosure. Specifically FIG. 6 shows the configuration of the medical observation system according to the present embodiment with a particular focus on a portion which extracts a movement of a treatment tool from sequentially captured images to detect a movement of an affected area where the treatment tool is held in a vicinity thereof and which executes various kinds of processing in accordance with a result of the detection. It should be noted that, in the Mowing description, the medical observation system shown in FIG. 6 is also referred to as a “medical observation system 3” for convenience's sake.

As shown in FIG. 6, the medical observation system 3 includes a control unit 301, an imaging portion 303, a detecting portion 305, and an output portion 307. For example, the imaging portion 303 can correspond to the imaging unit 203 (and eventually the imaging elements 215 and 217) shown in FIG. 4. In addition, the detecting portion 305 can correspond to the sensor driver 205 shown in FIG. 4. Furthermore, the output portion 307 can correspond to the output portion 209 shown in FIG. 4. Therefore, a detailed description of the imaging portion 303, the detecting portion 305, and the output portion 307 will be omitted.

The control unit 301 can correspond to the control unit 201 shown in FIG. 4. As shown in FIG. 6, the control unit 301 includes an image analyzing portion 309, a vibration detecting portion 311, an imaging control portion 313, an image processing portion 315, and an output control portion 817.

The image analyzing portion 309 acquires images sequentially captured by the imaging portion 303 and, by performing image analysis on the images, extracts a predetermined object (for example, a predetermined treatment tool such as a clip) having been captured in the images. As a specific example, the image analyzing portion 309 may calculate a feature amount of the acquired imaged and extract a portion having a predetermined feature from the images as a portion corresponding to a target object. It is needless to say that the example described above is merely an example and, as long as a predetermined object (a predetermined treatment tool) can be extracted from images having been sequentially captured by the imaging portion 303, a method thereof is not particularly limited. In the following description, images sequentially captured by the imaging portion 303 may also be simply referred to as a “captured image” for convenience's sake.

In addition, the image analyzing portion 309 may extract a predetermined affected area (for example, an affected area that is an observation object) from a captured image. In this case, the image analyzing portion 309 may extract a portion having a feature of an affected area to be an object from the captured image as a portion corresponding to the affected area. In addition, the image analyzing portion 309 outputs the captured image and an analysis result of the captured image (in other words, an extraction result of an object captured in the image) to the vibration detecting portion 311.

The vibration detecting portion 311 acquires the captured image and the analysis result of the captured image from the image analyzing portion 309. Based on the analysis result of the captured image, the vibration detecting portion 311 extracts a movement of a predetermined object (for example, vibration of a treatment tool) having been captured in the captured image. In addition, based on an extraction result of a movement of the predetermined object, the vibration detecting portion 311 may detect a movement of an affected area where the object is held in a vicinity thereof As a specific example, by detecting a movement of a clip used in a clipping surgery, the vibration detecting portion 311 may detect a movement of an aneurysm of which a neck or the like is being clipped by a clip. In addition, in this case, the vibration detecting portion 311 may detect a movement of an aneurysm to which a clip is being applied by taking into consideration a position where the clip is applied, a direction of the clip, and the like.

In addition, with respect to extracting a movement of a predetermined object such as a treatment tool and detecting a movement of an affected area based on an extraction result of the movement of the object, the vibration detecting portion 311 may use a detection result of vibration (for example, a detection result of a movement of the imaging portion 303 or a detection result of a movement of a portion of a patient) by the detecting portion 305. As a specific example, the vibration detecting portion 311 may extract a movement of a predetermined object such as a clip after correcting a blur (for example, a camera-shake) attributable to a movement of the imaging portion 303 using a detection result of the movement of the imaging portion 303 by the detecting portion 305. In a similar manner, the vibration detecting portion 311 may extract a movement of a predetermined object such as a clip after correcting a blur of an image attributable to a movement of a portion of a patient using a detection result of the movement of the portion by the detecting portion 305.

The vibration detecting portion 311 outputs the acquired captured image to at least any of the image processing portion 315 and the output control portion 317. In addition, for example, the vibration detecting portion 311 may output information related to a detection result of a movement of an affected area based on an analysis result of the captured image to at least any of the imaging control portion 313, the image processing portion 315, and the output control portion 317.

The imaging control portion 313 controls operations of the imaging portion 303. As a specific example, the imaging control portion 313 may control an operation for capturing an image by the imaging portion 303 in accordance with various conditions (for example, imaging conditions such as a shutter speed, an aperture, and a white balance) set via a predetermined input portion (not illustrated).

In addition, the imaging control portion 313 may acquire information related to a detection result of a movement of an affected area from the vibration detecting portion 311 and control operations of the imaging portion 303 based on the information. For example, the imaging control portion 313 may control imaging conditions related to imaging by the imaging portion 303 such as a shutter speed, an aperture, and a gain in accordance with a magnitude of a detected movement of an affected area. As a specific example, when a movement of an affected area is detected, the imaging control portion 313 may control a shutter speed such that, the greater a magnitude of the movement of the affected area, the higher the shutter speed by opening the aperture and increasing an amount of captured light. In addition, as another example, when a movement of an affected area is detected, the imaging control portion 313 may control the shutter speed so as to increase the shutter speed after increasing gain to enhance sensitivity of the imaging elements.

The image processing portion 315 performs various kinds of image processing on a captured image. As a specific example, the image processing portion 315 may correct lightness, a contrast, a tone, or the like of the captured image. In addition, the image processing portion 315 may generate an enlarged image of an affected area by cutting out and enlarging a part of the captured image (in other words, by performing digital zoom processing). Furthermore, the image processing portion 315 may perform image processing with respect to the captured image based on an instruction input via a predetermined input portion (not illustrated).

In addition, the image processing portion 315 may acquire information related to a detection result of a movement of an affected area from the vibration detecting portion 311 and perform image processing on the captured image based on the information. As a specific example, based on a detection result of a movement of an affected area, the image processing portion 315 may generate an image obtained by correcting a blur of the affected area (for example, a subject blur) that has become evident in a captured image (in other words, an image in which a blur of the affected area has been suppressed).

As described above, the image processing portion 315 performs image processing on a captured image and outputs the captured image after the image processing to the output control portion 317.

The output control portion 317 presents various kinds of information by outputting the information to the output portion 307. For example, the output control portion 317 may acquire a captured image and output the captured image to the output portion 307. In addition, the output control portion 317 may acquire the captured image having been subjected to image processing (hereinafter, also referred to as an “image after image processing”) from the image processing portion 315 and output the image after image processing to the output portion 307. Furthermore, as another example, the output control portion 317 may present display information indicating a flow area of interest, notification information such as a message or a warning, and the like by superimposing the information on an image.

In addition, the output control portion 317 may generate a screen (in other words, an image) presenting a plurality of types of information and present the plurality of types of information by outputting the screen to the output portion 307. As a specific example, the output control portion 317 may generate a screen presenting a captured image and an image after image processing and output the screen to the output portion 307. In this case, the output control portion 317 may generate a screen on which the captured image and the image after image processing are presented side-by-side. As another example, the output control portion 317 may generate a so-called PIP (Picture in Picture) image in which one of a captured image and an image after image processing is superimposed on a part of the other image. In this manner, the output control portion 317 may present a captured image and an image after image processing by associating the images with each other and, in this case, a presentation mode of the images (in other words, a method of associating the images with each other) is not particularly limited.

In addition, the output control portion 317 may acquire information related to a detection result of a movement of an affected area from the vibration detecting portion 311 and control output of various kinds of information to the output portion 307 based on the information. As a specific example, when a movement of an affected area is detected, the output control portion 317 may display a warning on the output portion 307 when a magnitude of the movement of the affected area is equal to or greater than a threshold. In addition, the output control portion 317 may selectively switch among information (for example, notification information such as a warning or a message) to be displayed on the output portion 307 in accordance with a magnitude of the movement of the affected area.

The functional configuration described above is merely an example and the functional configuration of the medical observation system is not necessarily limited to the example shown in FIG. 6 as long as operations of the respective components described above can be realized. As a specific example, at least any of the imaging portion 303, the detecting portion 305, and the output portion 307 may be integrally configured with the control unit 301. As another example, a part of functions of the control unit 301 may be provided outside of the control unit 301. In addition, at least a part of the functions of the control unit 301 may be realized by a cooperative operation of a plurality of apparatuses. Furthermore, a part of the configuration of the medical observation system according to the present embodiment may be modified or other components may be separately added without deviating from the fundamental concept of the technical features of the medical observation system described earlier.

It should be noted that an apparatus including a component corresponding to the control unit 301 shown in FIG. 6 corresponds to an example of the “medical observation apparatus”. In addition, the vibration detecting portion 311 corresponds to an example of the “detecting portion” that detects a movement of an affected area. Furthermore, as the imaging control portion 318, image processing portion 315, and output control portion 317, a component that executes or controls various kinds of processing (in particular, processing related to observation of an affected area) in accordance with a detection result of a movement of the affected area corresponds to an example of the “control portion”.

This concludes the description with reference to FIG. 3 of an example of a functional configuration of the medical observation system according to the embodiment of the present disclosure with a particular focus on an example of a functional configuration of a control unit that controls operations of various components of the medical observation system.

### 3.4 PROCESSING

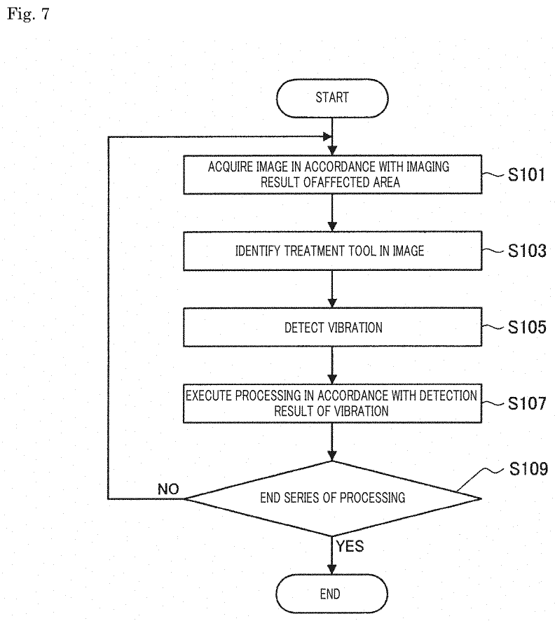

Next, an example of a flow of a series of processing by the medical observation system according to the embodiment of the present disclosure will be described with a particular focus on a flow of processing by which the control unit 301 shown in FIG. 6 detects a movement of an affected area and controls various operations based on a result of the detection. For example, FIG. 7 is a flow chart showing an example of a flow of a series of processing by the medical observation system according to the embodiment of the present disclosure.

First, the control unit 301 (the image analyzing portion 309) acquires images (in other words, captured images) of an affected area having been sequentially captured by the imaging portion 803 (S101) and, by performing image analysis on the images, identifies a predetermined treatment tool (for example, a clip) having been captured in the images (S103). In other words, based on an image analysis performed with respect to the captured images, the control unit 301 extracts a predetermined treatment tool from the captured images.

Next, by extracting a movement of the predetermined treatment tool based on an extraction result of the treatment tool from the captured images, the control unit 301 (the vibration detecting portion 311) detects a movement (for example, vibration) of an affected area where the treatment tool is held in a vicinity thereof (S105).

The control unit 301 controls various kinds of processing related to observation of an affected area in accordance with a detection result of a movement (vibration or the like) of the affected area (S107). For example, the control unit 301 (the imaging control portion 313) may control an operation related to imaging of the affected area by the imaging portion 303 in accordance with a detection result of a movement of the affected area. In addition, as another example, the control unit 301 (the image processing portion 315) may perform predetermined image processing with respect to a captured image based on a detection result of a movement of the affected area. Furthermore, as another example, the control unit 301 (the output control portion 317) may present various kinds of information via the output portion 307 in accordance with a detection result of a movement of the affected area.

As described above, unless an instruction to end the series of processing is issued (NO in S109), the control unit 301 sequentially executes processing denoted by the reference signs S101 to S107. In addition, once an instruction to end the series of processing is issued (YES in S109), the control unit 301 ends the execution of the processing denoted by the reference signs S101 to S107.

This concludes the description with reference to FIG. 7 of an example of a flow of a series of processing by the medical observation system according to the embodiment of the present disclosure with a particular focus on a flow of processing by which the control unit 301 shown in FIG. 6 detects a movement of an affected area and controls various operations based on a result of the detection.

### 3.5 EXAMPLES

Next, as Examples, examples of control of processing related to observation of an affected area based on a detection result of a movement of the affected area by the medical observation system according to the embodiment of the present disclosure will be described.

### Example 1: Example of Image Processing

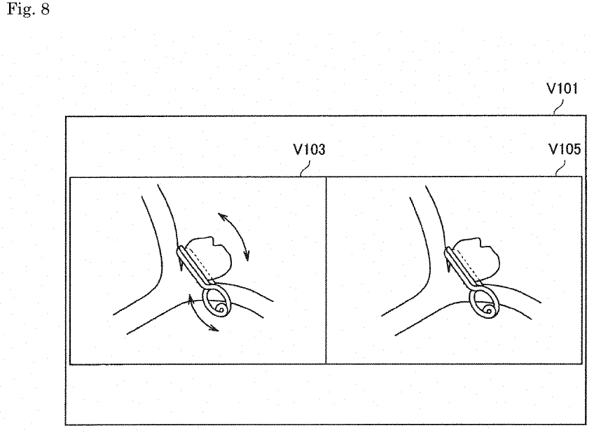

First, as Example 1, an example of performing image processing with respect to a captured image based on a detection result of a movement of an affected area will be described. For example, FIG. 8 is an explanatory diagram for explaining an example of control according to Example 1 and represents an example of performing image processing with respect to a captured image based on a detection result of a movement of an affected area, Specifically FIG. 8 represents an example of a case where image processing is performed with respect to a captured image of an affected area (an aneurysm) when assuming a situation where an observation of the affected area is performed by applying a clip as in a clipping surgery of an unruptured cerebral aneurysm.

As described earlier, a movement such as vibration exhibited by an affected area (for example, an aneurysm) of a blood vessel or the like accompanying pulsation may make an accurate observation difficult. Therefore, for example, by correcting a movement (for example, a subject blur) of an affected area in an image using a detection result of a movement of the affected area and generating an image in which a blur of the affected area has been suppressed (and eventually an image in which a presentation position of the affected area is fixed in the image), an effect of making the affected area more accurately observable can be expected. In this case, for example, by controlling a position where a part of the captured image including the affected area is to be cut out from the captured image based on a detection result of a movement of the affected area so as to cancel the movement of the affected area, an image in which a blur of the affected area has been canceled can be generated.

In addition, when image processing is performed with respect to a captured image, the image prior to the image processing and an image after the image processing may be presented in association with each other. For example, the example shown in FIG. 8 presents a screen V101 on which both a corrected image V105 created by suppressing (correcting) a blur of an affected area in an image through image processing and a captured image V103 (in other words, a so-called live image) before the image processing is performed are presented side-by-side. In this manner, by presenting the corrected image V105 created by suppressing a blur of an affected area in an image, a fine movement, a change, or the like of the affected area can be more accurately observed. An image to be presented as the screen V101 may be selectively switched to another image. For example, as shown in FIG. 8, a screen presenting both the captured image V103 and the corrected image V105 and a screen presenting only one of the captured image V103 and the corrected image V105 may be selectively switched. In addition, as another example, a so-called PIP image in which one of the captured image V103 and the corrected image V105 is superimposed on a part of the other image may be presented as the screen V101.

### Example 2: Example of Information Presentation

Next, as Example 2, an example of presenting various kinds of information in accordance with a detection result of a movement of an affected area will be described.

For example, in a situation where vibration of an affected area is large, even if an observation, a measurement, or the like of the affected area is performed, it can be assumed that obtaining an accurate result is difficult. Therefore, when such a situation is assumed and the movement of the affected area is large (for example, when a magnitude of the movement of the affected area is equal to or greater than a threshold), suppression of performance of an Observation or a measurement of the affected area may be prompted by presenting a warning.

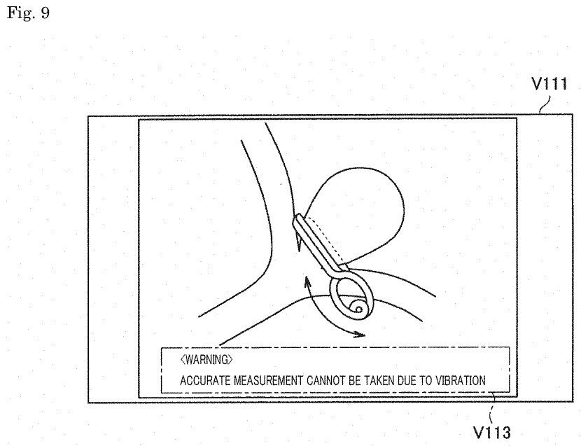

For example, FIG. 9 is an explanatory diagram for explaining an example of control according to Example 2 and represents an example of presenting information in accordance with a detection result of a movement of an affected area. Specifically in the example shown in FIG. 9, a magnitude of a movement of the affected area (an aneurysm) is equal to or greater than a threshold and a warning prompting suppression of performance of a measurement is presented as display information V113 on a screen V111 that presents a captured image.

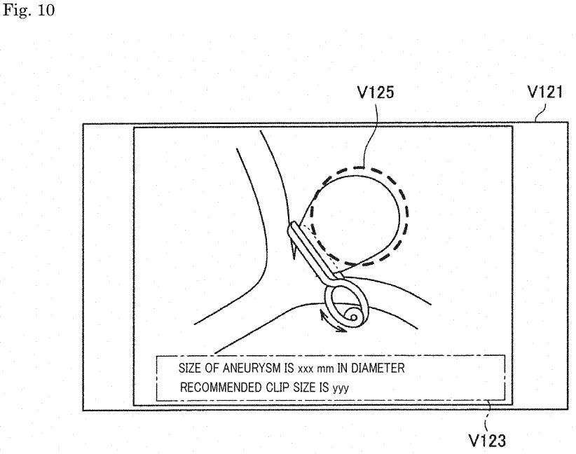

In addition, as another example, an operation related to presentation of information may be controlled in accordance with a magnitude of a movement of the affected area. For example, FIG. 10 is an explanatory diagram for explaining another example of control according to Example 2 and represents another example of presenting information in accordance with a detection result of a movement of an affected area. In the example shown in FIG. 10, when a movement of the affected area is small (for example, when a magnitude of the movement of the affected area is smaller than a threshold), information related to a procedure being performed (for example, information for supporting a procedure by an operator) is presented. Specifically, a size of an affected area (an aneurysm) (for example, a size of a dome of an aneurysm denoted by a reference sign V125) is measured based on image analysis or the like and a measurement result of the size is presented as display information V115 on a screen V121 that presents a captured image. It should be noted that a size of an affected area can be calculated based on, for example, information on a size in a captured image of an affected area extracted from the captured image or imaging conditions (for example, a focal length) of the captured image.

### Example 3: Example of Control of Operation Related to Imaging

Next, as Example 3, an example of controlling an operation related to imaging by the imaging portion based on a detection result of a movement of an affected area will be described.

Specifically, in a situation where an affected area to be an observation object is exhibiting a movement such as vibration, there is a possibility that so-called subject blur may occur. Such a subject blur becomes more evident as, for example, exposure time increases (in other words, as a shutter speed decreases). Therefore, for example, by controlling exposure tune such that the greater a magnitude of a movement of the affected area, the shorter the exposure time (in other words, the higher the shutter speed), an effect of a subject blur can be further reduced. On the other hand, an amount of light tends to decrease as the exposure time becomes shorter. Therefore, for example, when exposure time has been shortened, an amount of captured light may be further increased by, for example, opening an aperture. In addition, as another example, when exposure time has been shortened, a drop in lightness of a captured image that accompanies a drop in amount of captured light may be interpolated by increasing gain (in other words, improving imaging sensitivity).

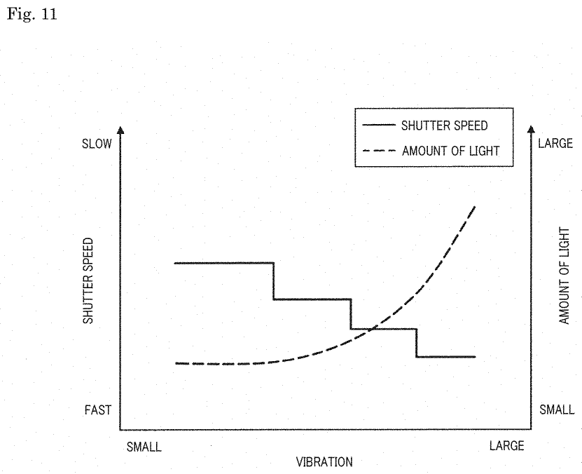

For example, FIG. 11 is an explanatory diagram for explaining an example of control according to Example 3 and represents an example of controlling an operation related to imaging by an imaging portion in accordance with a detection result of a movement of an affected area. In the example shown in FIG. 11, an abscissa represents a magnitude of vibration of an affected area (in other words, a magnitude of a movement of the affected area). In addition, in the example shown in FIG. 11, an example of a relationship between a magnitude of vibration of an affected area and each of a shutter speed and an amount of light is shown. In other words, in the example shown in FIG. 11, an ordinate of a graph representing a relationship between a magnitude of vibration of an affected area and a shutter speed represents a magnitude of the shutter speed. Furthermore, an ordinate of a graph representing a relationship between a magnitude of vibration of an affected area and a magnitude of an amount of light (in other words, an amount by which an aperture is opened) represents a magnitude of the amount of light.

In other words, in the example shown in FIG. 11, as described earlier, control is performed such that the larger the vibration of an affected area, the higher the shutter speed and the larger the amount of light (in other words, the more open the aperture). In addition, as another example, a gain (in other words, imaging sensitivity) to be applied to an imaging result may be controlled instead of a magnitude of an amount of captured light. In this case, control for increasing gain may be applied in place of control for increasing an amount of light. In other words, control may be performed such that the larger vibration of an affected area, the higher the shutter speed and the larger the gain (in other words, the higher the imaging sensitivity). In addition, both control of an amount of captured light and control of gain may be performed. Furthermore, an amount of irradiating light from a light source may be controlled so as to increase while keeping aperture and gain constant. Due to the control described above, since conditions related to imaging of an affected area by an imaging portion are controlled in accordance with a magnitude of a movement of the affected area, an occurrence of a situation where vibration of the affected area makes observation of the affected area difficult can be further suppressed.

### 4. EXAMPLE OF HARDWARE CONFIGURATION

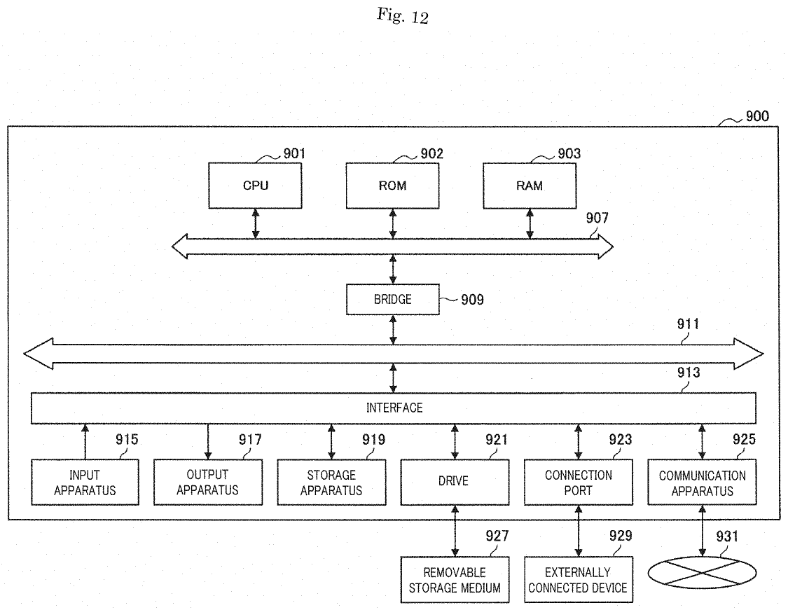

Next, with reference to FIG. 12, an example of a hardware configuration of an information processing apparatus (for example, the control unit 201 shown in FIG. 4 or the control unit 310 shown in FIG. 5) that executes various kinds of processing in the medical observation system according to the present embodiment will be described in detail, FIG. 12 is a functional block diagram showing a configuration example of the hardware configuration of the information processing apparatus that constitutes the medical observation system according to the embodiment of the present disclosure.

An information processing apparatus 900 that constitutes the medical observation system according to the present embodiment mainly includes a CPU 901, a ROM (Read Only Memory) 908, and a RAM (Random Access Memory) 905. In addition, the information processing apparatus 900 further includes a host bus 907, a bridge 909, an external bus 911, an interface 913, an input apparatus 915, an output apparatus 917, a storage apparatus 919, a drive 921, a connection port 923, and a communication apparatus 925.

The CPU 901 functions as an arithmetic processing apparatus and a control apparatus and, in accordance with various programs recorded in the ROM 903, the RAM 905, the storage apparatus 919, or a removable recording medium 927, controls all operations or a part thereof inside the information processing apparatus 900. The ROM 903 stores programs, arithmetic parameters, and the like to be used by the CPU 901. The RAM 905 primarily stores programs to be used by the CPU 901, parameters that change from time to time when the programs are being executed, and the like. The CPU 901, the ROM 903, and the RAM 905 are connected to each other by the host bus 907 that is constituted by an internal bus such as a CPU bus. It should be noted that the respective components of the control unit 301 shown in FIG. 6 or, in other words, the image analyzing portion 309, the vibration detecting portion 311, the imaging control portion 313, the image processing portion 315, and the output control portion 317 can be realized by the CPU 901.

The host bus 907 is connected to the external bus 911 that is a PCI (Peripheral Component Interconnect/Interface) bus or the like via the bridge 909. In addition, the input apparatus 915, the output apparatus 917, the storage apparatus 919, the drive 921, the connection port 923, and the communication apparatus 925 are connected to the external bus 911 via the interface 913.

The input apparatus 915 is operating means to be operated by the user of which examples include a mouse, a keyboard, a touch panel, a button, a switch, a lever, and a pedal. In addition, the input apparatus 915 may be remote-controlled means (a so-called remote controller) that utilizes infrared light or other radio waves or an externally-connected device 929 such as a mobile phone or a PDA that accommodates operations of the information processing apparatus 900. Furthermore, for example, the input apparatus 915 is constituted by an input control circuit or the like which generates an input signal based on information input by the user using the operating means described above and which outputs the generated input signal to the CPU 901. By operating the input apparatus 915, the user of the information processing apparatus 900 can input various kinds of data and issue instructions to perform processing operations with respect to the information processing apparatus 900.

The output apparatus 917 is constituted by an apparatus capable of visually or auditorily notifying the user of acquired information. Examples of such an apparatus include display apparatuses such as a liquid crystal display apparatus, an organic EL (Electro Luminescence) display apparatus, a CRT (Cathode Ray Tube) a display apparatus, a plasma display apparatus, and a lamp, audio output apparatuses such as a speaker and headphones, and printer apparatuses. For example, the output apparatus 917 outputs results obtained through various kinds of processing performed by the information processing apparatus 900. Specifically, the display apparatus outputs, in the form of a text or an image, results obtained by various kinds of processing performed by the information processing apparatus 900. On the other hand, the audio output apparatus converts an audio signal constituted by reproduced speech data, acoustic data, or the like into an analog signal and outputs the converted analog signal. The output portion 307 shown in FIG. 6 can be realized by the output apparatus 917.

The storage apparatus 919 is an apparatus for data storage having been configured as an example of a storage portion of the information processing apparatus 900. For example, the storage apparatus 919 is constituted by a magnetic storage portion device such as an HDD (Hard Disk Drive), a semiconductor storage device, an optical storage device, a magneto-optical storage device, or the like. The storage apparatus 919 stores programs to be executed by the CPU 901, various kinds of data, and the like.

The drive 921 is recording medium reader/writer and is built into or externally attached to the information processing apparatus 900. The drive 921 reads information recorded in the removable recording medium 927 that is a magnetic disk, an optical disk, a magneto optical disk, a semiconductor memory, or the like being mounted to the drive 921 and outputs the read information to the RAM 905. In addition, the drive 921 can also write records into the removable recording medium 927 that is a magnetic disk, an optical disk, a magneto optical disk, a semiconductor memory, or the like being mounted to the drive 921. For example, the removable recording medium 927 is a DVD medium, an HD-DVD medium, a Blu-ray (a registered trademark) medium, or the like. In addition, the removable recording medium 927 may be a compact flash (a registered trademark) (CF: Compact Flash (a registered trademark)), a flash memory, an SD memory card (a Secure Digital memory card), or the like. Furthermore, the removable recording medium 927 may be, for example, an IC card (an Integrated Circuit card) mounted with a contactless IC chip, an electronic device, or the like.

The connection port 923 is a port for directly connecting to the information processing apparatus 900. Examples of the connection port 928 include a USB (Universal Serial Bus) port, an IEEE 1394 port, and a SCSI (Small Computer System interface) port. Other examples of the connection port 923 include an RS-232C port, an optical audio terminal, and an HDMI (a registered trademark) (High-Definition Multimedia Interface) port. By connecting the externally-connected device 929 to the connection port 923, the information processing apparatus 900 can directly acquire various kinds of data from the externally-connected device 929 and provide the externally-connected device 929 with various kinds of data.

The communication apparatus 925 is, for example, a communication interface constituted by a communication device or the like for connecting to a communication network (a network) 931. For example, the communication apparatus 925 is a communication card for a wired or wireless LAN (Local Area Network), Bluetooth (a registered trademark), or WUSB (Wireless USB). In addition, the communication apparatus 925 may be a router for optical communication, a router for ADSL (Asymmetric Digital Subscriber Line), or a modem or the like for various kinds of communication. For example, the communication apparatus 925 is capable of transmitting and receiving signals and the like in conformity to a predetermined protocol such as TCP/IP to and from the Internet or another communication device. In addition, a communication network 931 to be connected to the communication apparatus 925 is constituted by a network or the like connected in a wired or wireless manner and may be constituted by for example, the Internet, a domestic LAN, infrared communication, radio wave communication, or satellite communication.

This concludes the description of an example of a hardware configuration capable of realizing functions of the information processing apparatus 900 that constitutes the medical observation system according to the embodiment of the present disclosure. The respective components described above may be constructed using generic members or constituted by hardware having been specialized for the functions of the respective components. Therefore, the hardware configuration to be utilized can be modified as deemed appropriate in accordance with a technological level at the time of implementation of the present embodiment, Although not illustrated in FIG. 12, various components corresponding to the information processing apparatus 900 which constitutes the medical observation system are obviously included.

A computer program for realizing the various functions of the information processing apparatus 900 that constitutes the medical observation system according to the present embodiment as described above can be fabricated and mounted on a personal computer or the like. In addition, a computer-readable recording medium storing such a computer program can also be provided. The recording medium is, for example, a magnetic disk, an optical disk, a magneto-optical disk, or a flash memory. Furthermore, the computer program described above may be distributed via a network or the like without using a recording medium. In addition, the number of computers to execute the computer program is not particularly limited. For example, a plurality of computers (for example, a plurality of servers) may execute the computer program by cooperating with each other.

### 5. APPLICATIONS

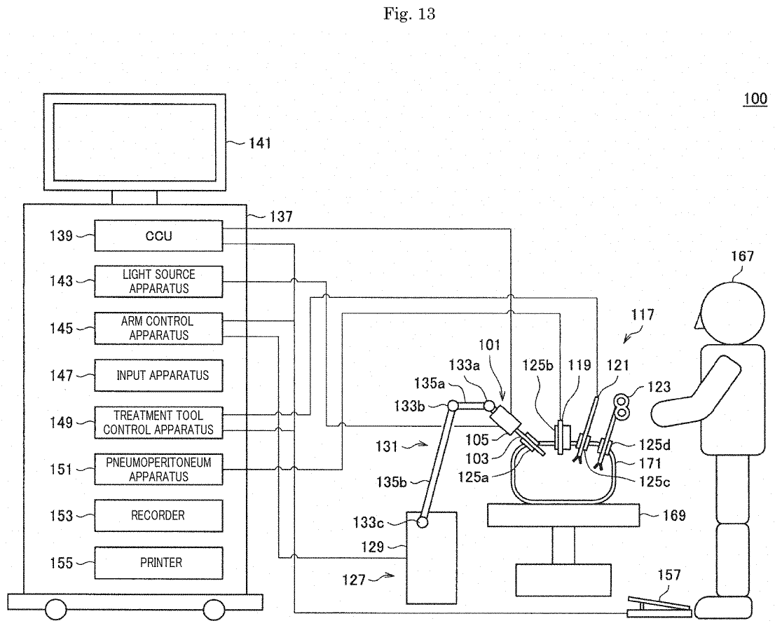

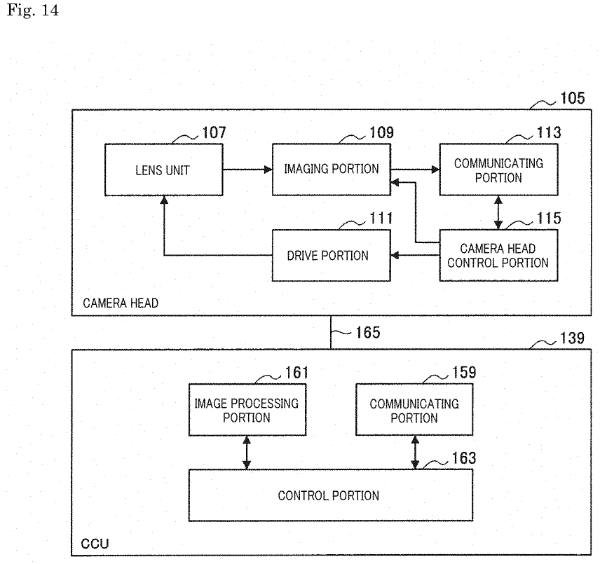

Next, as an application of the medical observation system according to the embodiment of the present disclosure, an example in which the medical observation system is configured as an endoscopic surgery system including a microscope unit will be described with reference to FIGS. 13 and 14.

FIGS. 13 and 14 are explanatory diagrams for explaining applications of the medical observation system according to the embodiment of the present disclosure and represent an example of a schematic configuration of an endoscopic surgery system.

For example, FIG. 13 is a diagram showing an example of a schematic configuration of an endoscopic surgery system to which the technique according to the present disclosure can he applied. FIG. 13 shows a situation where an operator (a medical doctor) 167 is performing a surgery on a patient 171 on a patient bed 169 using an endoscopic surgery system 100. As illustrated, the endoscopic surgery system 100 is constituted by an endoscope 101, other surgical tools 117, a support arm apparatus 127 for supporting the endoscope 101, and a cart 137 mounted with various apparatuses for an endoscopic surgery.

In an endoscopic surgery instead of performing laparotomy by making an incision in the abdominal wall, cylindrical hole-opening instruments called trocars 125a to 125d are punctured through the abdominal wall in plurality. In addition, a lens tube 103 of the endoscope 101 and other surgical tools 117 are inserted into a body cavity of the patient 171 through the trocars 125a to 125d. In the illustrated example, a pneumoperitoneum tube 119, an energized treatment tool 121, and forceps 123 are inserted into the body cavity of the patient 171 as the other surgical tools 117. Furthermore, the energized treatment tool 121 is a treatment tool for performing tissue dissection and resection, sealing of a blood vessel, and the like using a high-frequency current or ultrasonic vibrations. However, the illustrated surgical tools 117 are merely examples and various surgical tools generally used in an endoscopic surgery such as forceps and a retractor may be used as the surgical tools 117.

An image of an operative site inside the body cavity of the patient 171 having been photographed by the endoscope 101 is displayed on a display apparatus 141. For example, the operator 167 performs treatment such as an excision of an affected part using the energized treatment tool 121 or the forceps 123 while viewing the image of the operative site displayed on the display apparatus 141 in real-time. Although not illustrated, the pneumoperitoneum tube 119, the energized treatment tool 121, and the forceps 123 are supported by the operator 167, an assistant, or the like during the surgery.

(Support Arm Apparatus)

The support arm apparatus 127 includes an arm portion 131 that stretches from a base portion 129. In the illustrated example, the arm portion 131 is constituted by joint portions 133a, 133b, and 133c and links 135a and 135b and is driven under control of an arm control apparatus 145. The arm portion 131 supports the endoscope 101 and controls a position and a posture thereof. Accordingly stable fixation of the position of the endoscope 101 can be realized.

(Endoscope)

The endoscope 101 is constituted by a lens tube 103 of which a region of a predetermined length from a tip is to be inserted into a body cavity of the patient 171 and a camera head 105 that is connected to a base end of the lens tube 103. While the illustrated example represents the endoscope 101 that is configured as a so-called rigid scope having a rigid lens tube 103, alternatively the endoscope 101 may be configured as a so-called flexible scope having a flexible lens tube 103. It should be noted that the camera head 105 or the endoscope 101 including the camera head 105 corresponds to an example of the “medical observation apparatus”.

An opening fitted with an objective lens is provided at the tip of the lens tube 103. A light source apparatus 143 is connected to the endoscope 101, and light generated by the light source apparatus 143 is guided to the tip of the lens tube 103 by a light guide that is provided so as to extend inside the lens tube 103 and irradiated via the objective lens toward an observation object (in other words, an imaging object) inside the body cavity of the patient 171. It should be noted that the endoscope 101 may be a forward-viewing endoscope, an angled endoscope, or a lateral-viewing endoscope.

An optical system and an imaging element are provided inside the camera head 105, and reflected light (observation light) from the observation target is collected on the imaging element by the optical system. The observation light is photoelectrically converted by the imaging element and an electric signal corresponding to the observation light or, in other words, an image signal corresponding to an observation image is generated. The image signal is transmitted to a camera control unit (CCU) 139 as RAW data. The camera head 105 is mounted with a function for adjusting a magnification and a focal length by suitably driving an optical system thereof.

For example, the camera head 105 may be provided with a plurality of imaging elements in order to accommodate stereoscopic viewing (3D display) or the like. In this case, relay optical systems are provided in plurality inside the lens tube 103 in order to guide observation light to each of the plurality of imaging elements.

(Various Apparatuses Mounted on Cart)

The CCU 139 is constituted by a CPU (Central Processing Unit), a GPU (Graphics Processing Unit), and the like and comprehensively controls operations of the endoscope 101 and the display apparatus 141. Specifically, with respect to an image signal received from the camera head 105, the CCU 139 performs various kinds of image processing for displaying an image based on the image signal such as development processing (demosaicking). The CCU 139 provides the display apparatus 141 with the image signal having been subjected to the image processing. In addition, the CCU 139 transmits a control signal to the camera head 105 to control drive of the camera head 105. The control signal can include information related to imaging conditions such as a magnification and a focal length.

Under the control of the CCU 139, the display apparatus 141 displays an image based on the image signal having been subject to image processing by the CCU 139. For example, when the endoscope 101 accommodates high-resolution photography such as 4K (3840 horizontal pixels×2160 vertical pixels) or 8K (7680 horizontal pixels×4320 vertical pixels) and/or accommodates 3D display, a corresponding display apparatus capable of high-resolution display and/or a corresponding display apparatus capable of 3D display can be used as the display apparatus 141. When the endoscope 101 accommodates high-resolution photography such as 4K or 8K, a further sense of immersion can be obtained by using a display apparatus with a size of 55 inches or larger as the display apparatus 141. In addition, a plurality of display apparatuses 141 with different resolutions and sizes may be provided depending on the intended use.

For example, the light source apparatus 143 is constituted by a light source such as an LED (light emitting diode) and supplies the endoscope 101 with irradiating light when photographing an operative site.

For example, the arm control apparatus 145 is constituted by a processor such as a CPU and, by operating in accordance with a predetermined program, controls drive of the arm portion 131 of the support arm apparatus 127 in accordance with a predetermined control system.

An input apparatus 147 is an input interface with respect to the endoscopic surgery system 100. The user can input various kinds of information and various instructions to the endoscopic surgery system 100 via the input apparatus 147. For example, via the input apparatus 147, the user inputs various kinds of information related to a surgery such as physical information of a patient and information on an operative method of the surgery. In addition, for example, via the input apparatus 147, the user inputs an instruction to the effect of driving the arm portion 131, an instruction to the effect of changing imaging conditions (a type of irradiating light, a magnification, a focal length, and the like) of the endoscope 101, an instruction to the effect of driving the energized treatment tool 121, and the like.

A type of the input apparatus 147 is not limited and the input apparatus 147 may be various known input apparatuses. As the input apparatus 147, for example, a mouse, a keyboard, a touch panel, a switch, a foot switch 157 and/or a lever, or the like can be applied. When a touch panel is used as the input apparatus 147, the touch panel may be provided on a display surface of the display apparatus 141.

Alternatively, the input apparatus 147 may be a sensor built into a device to be worn by the user such as an eyeglass-type wearable device or an HMD (Head Mounted Display) and, in this case, various kinds of input are performed in accordance with a movement or a line of sight of the user as detected by the sensor. In addition, the input apparatus 147 includes a camera capable of detecting a movement of the user, and various kinds of input are performed in accordance with a gesture or a line of sight of the user as detected from a video taken by the camera. Furthermore, the input apparatus 147 includes a microphone capable of picking up the user's voice, and various kinds of input are performed by sound via the microphone. In this manner, by configuring the input apparatus 147 so that various kinds of information can be input in a contactless manner, in particular, the user (for example, the operator 167) belonging to a clean area can now operate a device belonging to an unclean area in a contactless manner. In addition, since the user can operate a device without letting go of a surgical tool being held by the user, convenience of the user improves.

A treatment tool control apparatus 149 controls drive of the energized treatment; tool 121 for cauterizing or incising tissue, sealing a blood vessel, or the like. A pneumoperitoneum apparatus 151 feeds gas into the body cavity of the patient 171 via the pneumoperitoneum tube 119 in order to expand the body cavity for the purpose of securing a field of view of the endoscope 101 and securing a work space for the operator. A recorder 153 is an apparatus that is capable of recording various kinds of information related to the surgery A printer 155 is an apparatus that is capable of printing various kinds of information related to the surgery in various formats such as a text, an image, and a graph.

Hereinafter, particularly characteristic components of the endoscopic surgery system 100 will be described in greater detail.

(Support Arm Apparatus)

The support arm apparatus 127 includes the base portion 129 that constitutes a base and the arm portion 131 that stretches from the base portion 129. While the arm portion 131 is constituted of a plurality of joint portions 133a, 133b, and 133c and a plurality of links 135a and 135b that; are coupled by the joint portion 133b in the illustrated example, FIG. 13 illustrates a construction of the arm portion 131 in a simplified manner for the sake of simplicity. In reality, to provide the arm portion 131 with a desired degree of freedom, shapes, numbers, and arrangements of the joint portions 133a to 133c and the links 135a and 135b, directions of rotational axes of the joint portions 133a to 133c, and the like can be suitably set. For example, the arm portion 131 can be preferably configured so as to have a degree of freedom of six degrees of freedom or more. Accordingly since the endoscope 101 can be freely moved within a movable range of the arm portion 131, the lens tube 103 of the endoscope 101 can be inserted into the body cavity of the patient 171 from a desired direction.

The joint portions 133a to 133c are provided with actuators, and the joint portions 133a to 133c are configured so as to be rotatable around predetermined rotational axes by drive of the actuators. Due to the drive of the actuators being controlled by the arm control apparatus 145, a rotation angle of each of the joint portions 133a to 133c is controlled and drive of the arm portion 131 is controlled. Accordingly control of a position and a posture of the endoscope 101 can be realized. In doing so, the arm control apparatus 145 is capable of controlling drive of the arm portion 131 by various known control systems such as power control and position control.

For example, by having the operator 167 perform suitable operation input via the input apparatus 147 (including the foot switch 157), the drive of the arm portion 131 may be suitably controlled by the arm control apparatus 145 in accordance with the operation input and the position and the posture of the endoscope 101 may be controlled. Due to the control, after moving the endoscope 101 at a tip of the arm portion 131 from an arbitrary position to another arbitrary position, the endoscope 101 can be supported in a fixed manner at the position after the movement. It should be noted that the arm portion 131 may be operated by a so-called master-slave system. In this case, the arm portion 131 can be remotely operated by the user via the input apparatus 147 installed at a location separated from an operating room.

In addition, when power control is applied, the arm control apparatus 145 may perform so-called power assist control in which, upon receiving an external force from the user, the actuators of the respective joint portions 133a to 133c are controlled so that the arm portion 131 moves smoothly along the external force. Accordingly when the user moves the arm portion 131 while directly touching the arm portion 131, the arm portion 131 can be moved with a relatively small force. Therefore, since the endoscope 101 can be moved more intuitively with a simpler operation, convenience of the user can be improved.

Generally, in endoscopic surgeries, the endoscope 101 has been supported by a medical doctor referred to as a scopist. In contrast, since the use of the support arm apparatus 127 enables the position of the endoscope 101 to be more reliably fixed without human intervention, an image of an operative site can be obtained in a stable manner and a surgery can be performed smoothly.

The arm control apparatus 145 need not necessarily be provided on the cart 137. In addition, the arm control apparatus 145 need not necessarily be a single apparatus. For example, the arm control apparatus 145 may be respectively provided on each of the joint portions 133a to 133c of the arm portion 131 of the support arm apparatus 127, and drive control of the arm portion 131 may be realized by having the plurality of arm control apparatuses 145 cooperate with each other.

(Light Source Apparatus)

The light source apparatus 143 supplies the endoscope 101 with irradiating light when photographing an operative site. For example, the light source apparatus 143 is constituted by a white light source that is an LED, a laser light source, or a combination thereof. In this case, when a white light source is constituted by a combination of RGB laser light sources, since output intensity and an output timing of each color (each wavelength) can be controlled with high accuracy, white balance of a captured image can be adjusted by the light source apparatus 143. In addition, in this case, by irradiating the observation target with laser light from each of the RGB laser light sources by time division and controlling drive of the imaging element of the camera head 105 in synchronization with the irradiation timings, an image corresponding to each of RGB can be captured by time division. According to the method, a color image can be obtained without providing the imaging element with a color filter.

In addition, drive of the light source apparatus 143 may be controlled so that intensity of output light changes at predetermined time intervals. By controlling drive of the imaging element of the camera head 105 in synchronization with a timing of change of the intensity of light to acquire images by time division and by compositing the images, an image with a high dynamic range with no so-called blocked-up shadows or blown-out highlights can be generated.

Furthermore, the light source apparatus 143 may be configured to be capable of supplying light of a predetermined wavelength band that corresponds to special light observation. In special light observation, for example, by utilizing wavelength dependency of absorption of light of body tissue to irradiate light with a narrower band than irradiating light (in other words, white light) during a normal observation, so-called narrow band light observation (Narrow Band Imaging) is performed in which predetermined tissue such as a capillary in a mucous membrane surface layer is photographed in high contrast. Alternatively, in special light observation, fluorescent observation in which an image is obtained by fluorescent light generated by irradiating excitation light may be performed. In fluorescent observation, body tissue can be irradiated with excitation light and fluorescent light from the body tissue can be observed (self-fluorescent observation), an agent such as indocyanine green (ICG) can be locally injected into body tissue and the body tissue may be irradiated with excitation light corresponding to a fluorescent light wavelength of the agent to obtain a fluorescent image, and the like. The light source apparatus 143 can be configured to be capable of supplying narrow band light and/or excitation light that accommodates such special light observation.

(Camera Head and CCU)

Functions of the camera head 105 of the endoscope 101 and the CCU 139 will be described in greater detail with reference to FIG. 14. FIG. 14 is a block diagram showing an example of functional configurations of the camera head 105 and the CCU 139 shown in FIG. 13.

With reference to FIG. 14, the camera head 105 has a lens unit 107, an imaging portion 109, a drive portion 111, a communication portion 113, and a camera head control portion 115 as functions thereof. In addition, the CCU 139 has a communication portion 159, an image processing portion 161, and a control portion 163 as functions thereof, The camera head 105 and the CCU 139 are connected by a transmission cable 165 so as to be capable of communicating in both directions.

First, a functional configuration of the camera head 105 will be described. The lens unit 107 is an optical system that is provided in a connecting portion with the lens tube 103. Observation light taken in from a tip of the lens tube 103 is guided to the camera head 105 and input to the lens unit 107. The lens unit 107 is constructed by combining a plurality of lenses including a zoom lens and a focus lens. Optical characteristics of the lens unit 107 are adjusted so that observation light converges on a light-receiving surface of the imaging element of the imaging portion 109, In addition, the zoom lens and the focus lens are configured such that positions on optical axes thereof are movable in order to adjust a magnification and a focus of a captured image.

The imaging portion 109 is constituted by an imaging element and is arranged in a stage subsequent to the lens unit 107. Observation light having passed through the lens unit 107 converges on a light-receiving surface of the imaging element and an image signal corresponding to an observed image is generated by photoelectric conversion. The image signal generated by the imaging portion 109 is provided to the communication portion 113.

As the imaging element that constitutes the imaging portion 109, for example, while a CMOS (Complementary Metal Oxide Semiconductor) type image sensor or a CCD (Charge Coupled Device) type image sensor capable of performing color photography having a Bayer array is used, a single-plate imaging element for black-and-white photography may also be used. A plurality of image sensors for black-and-white photography may be used. As the imaging element, for example, an imaging element capable of accommodating photography of a high-resolution image of 4K or higher may be used. Obtaining an image of an operative site at high resolution enables the operator 167 to assess a situation of the operative site in greater detail and enables the surgery to proceed more smoothly.

In addition, the imaging element constituting the imaging portion 109 may be configured to have a pair of imaging elements for respectively acquiring right-eye and left-eye image signals that correspond to 3D display. Performing 3D display enables an operator 167 to more accurately assess a depth of body tissue in the operative site. When the imaging portion 109 is constituted by a multi-plate imaging element, the lens unit 107 is also provided in plurality in correspondence with the respective imaging elements.

In addition, the imaging portion 109 need not necessarily be provided in the camera head 105. For example, the imaging portion 109 may be provided immediately behind an objective lens inside the lens tube 103.

The drive portion 111 is constituted by an actuator and, under control from the camera head control portion 115, moves the zoom lens and the focus lens of the lens unit 107 by a predetermined distance along an optical axis. Accordingly, a magnification and a focus of a captured image by the imaging portion 109 can be appropriately adjusted.

The communication portion 113 is constituted by a communication apparatus for transmitting and receiving various kinds of information to and from the CCU 139. The communication portion 118 transmits an image signal obtained from the imaging portion 109 to the CCU 139 via the transmission cable 165 as RAW data. In doing so, in order to display a captured image of the operative site with as little delay as possible, the image signal is favorably transmitted by optical communication. This is because, during a surgery, since the operator 167 performs the surgery while observing a state of an affected area through a captured image, it is required that a moving image of an operative site is displayed in real-time as much as possible in order to ensure that the surgery is performed in a safe and reliable manner. When optical communication is performed, the communication portion 113 is provided with a photoelectric conversion module that converts an electric signal into an optical signal. After an image signal is converted into an optical signal by the photoelectric conversion module, the converted signal is transmitted to the CCU 139 via the transmission cable 165.

In addition, the communication portion 113 receives a control signal for controlling drive of the camera head 105 from the CCU 139. For example, the control signal includes information related to imaging conditions such as information to the effect of designating a frame rate of a captured image, information to the effect of designating imaging conditions (a shutter speed, an aperture, a gain, and the like) during photography and/or information to the effect of designating a magnification and a focus of the captured image. The communication portion 113 provides the camera head control portion 115 with the received control signal. A control signal from the CCU 139 may also be transmitted by optical communication. In this case, the communication portion 113 is provided with a photoelectric conversion module that converts an optical signal into an electric signal, and after the control signal is converted into an electric signal by the photoelectric conversion module, the camera head control portion 115 is provided with the converted signal.

It should be noted that the imaging conditions such as a frame rate, an exposure value, a magnification, and a focus described above are automatically set, by the control portion 163 of the CCU 139 based on an acquired image signal. In other words, a so-called AE (Auto Exposure) function, an AF (Auto Focus) function, and an AWB (Auto White Balance) function are realized by the CCU 139 and the endoscope 101.

Based on the control signal from the CCU 139 received via the communication portion 113, the camera head control portion 115 controls drive of the camera head 105. For example, the camera head control portion 115 controls drive of the imaging element of the imaging portion 109 based on information to the effect of designating a frame rate of a captured image and/or information to the effect of designating a shutter speed and an aperture during imaging. In addition, for example, the camera head control portion 115 suitably moves the zoom lens and the focus lens of the lens unit 107 via the drive portion 111 based on information to the effect of designating a magnification and a focus of the captured image. The camera head control portion 115 may be further equipped with a function of storing information for identifying the lens tube 103 and the camera head 105.

It should be noted that, by arranging components such as the lens unit 107 and the imaging portion 109 in a sealed structure with high airtightness and high waterproofness, the camera head 105 can be imparted with resistance to autoclave sterilization processing.

Next, a functional configuration of the CCU 139 will be described. The communication portion 159 is constituted by a communication apparatus for transmitting and receiving various kinds of information to and from the camera head 105. The communication portion 159 receives an image signal transmitted from the camera head 105 via the transmission cable 165. In doing so, the image signal can preferably be transmitted by optical communication as described above. In this case, in order to accommodate optical communication, the communication portion 159 is provided with a photoelectric conversion module that converts an optical signal into an electric signal. The communication portion 159 provides the image processing portion 161 with the image signal having been converted into an electric signal.

In addition, the communication portion 159 transmits, to the camera head 105, a control signal for controlling drive of the camera head 105, The control signal may also be transmitted by optical communication.

The image processing portion 161 performs various kinds of image processing on an image signal transmitted from the camera head 105 that is RAW data. Examples of the image processing include various known types of signal processing such as development processing, image quality enhancement processing (band emphasis processing, super resolution processing, NR (Noise reduction) processing and/or camera-shake correction processing), and/or enlargement processing (electronic zoom processing). In addition, the image processing portion 161 performs detection processing with respect to an image signal for performing AE, AF, and AWB.

The image processing portion 161 is constituted by a processor such as a CPU or a GPU and the image processing and the detection processing described above can be performed as the processor operates according to a predetermined program. When the image processing portion 161 is constituted by a plurality of GPUs, the image processing portion 161 suitably divides information related to an image signal and performs image processing in parallel using the plurality of GPUs.

The control portion 163 performs various kinds of control related to imaging of an operative site by the endoscope 101 and display of a captured image thereof. For example, the control portion 163 generates a control signal for controlling drive of the camera head 105. In doing so, when imaging conditions have been input by the user, the control portion 163 generates the control signal based on the input by the user. Alternatively when the endoscope 101 is equipped with an AE function, an AF function, and an AWB function, the control portion 163 suitably calculates optimal exposure conditions, an optimal focal length, and an optimal white balance in accordance with a result of detection processing by the image processing portion 161 and generates the control signal.

In addition, based on an image signal having been subjected to image processing by the image processing portion 161, the control portion 163 causes the display apparatus 141 to display an image of an operative site. In doing so, the control portion 163 recognizes various kinds of objects inside the operative site image using various kinds of image recognition techniques. For example, by detecting a shape, a color, and the like of an edge of an object included in the operative site image, the control portion 163 can recognize surgical tools such as forceps, a specific biological site, a hemorrhage, and mist or the like when using the energized treatment tool 121. When causing the display apparatus 141 to display the operative site image, using a recognition result thereof, the control portion 163 causes various kinds of surgery support information to be displayed so as to be superimposed on the operative site image. Superimposing and displaying the surgery support information and presenting the same to the operator 167 enables the operator 167 to carry out the surgery in a safe and reliable manner.

The transmission cable 165 that connects the camera head 105 and the CCU 139 is an electric signal cable that accommodates communication of electric signals, an optical fiber that accommodates optical communication, or a composite cable thereof.

While communication is performed in a wired manner using the transmission cable 165 in the illustrated example, alternatively the communication between the camera head 105 and the CCU 139 may be performed in a wireless manner. When the communication between the camera head 105 and the CCU 139 is performed wirelessly, since there is no longer a need to lay the transmission cable 165 in an operating room, a situation where movement of medical personnel in the operating room is hindered by the transmission cable 165 can be eliminated.

This concludes the description of an example of the endoscopic surgery system 100 to which the technique according to the present disclosure can be applied. While the endoscopic surgery system 100 has been described as an example, systems to which the technique according to the present disclosure can be applied are not limited to this example. For example, the technique according to the present disclosure may be applied to an inspection soft endoscope system and a microscopic surgery system.

In addition to the above, the technique according to the present disclosure described above can be applied without deviating from the fundamental concept of the medical observation system according to the embodiment of the present disclosure. As a specific example, in addition to systems to which the endoscope described above or a surgical microscope is applied, the technique according to the present disclosure described above can be suitably applied to a system that enables an affected area to be observed by capturing an image of the affected area with an imaging apparatus in a desired mode.

In addition, it is needless to say that an observation method of an affected area and a procedure to be applied are also not particularly limited. For example, an observation method (a treatment method) of an aneurysm as an affected area to be an observation object is not limited to the clipping surgery described above and a method using a stent and a method using a flow diverter are known. Furthermore, treatment tools to be used may differ depending on the observation method or the procedure to be applied. Even in such cases, for example, as long as a treatment tool is held in a vicinity of an affected area, by applying the technique according to the present disclosure described above and extracting a movement of the treatment tool from sequentially captured images of the affected area, a movement of the affected area can be detected.

This concludes the description of an example in which the medical observation system according to the embodiment of the present disclosure is configured as an endoscopic surgery system with reference to FIGS. 13 and 14 as an application of the medical observation system.

### 6. CONCLUSION

As described above, a medical observation system according to an embodiment of the present disclosure includes an imaging portion, a detecting portion, and a control portion. The imaging portion captures images of an affected area. The detecting portion extracts a movement of a treatment tool held in a vicinity of the affected area based on images of the affected area having been sequentially captured by the imaging portion and detects a movement of the affected area based on a result of the extraction. The control portion controls processing related to observation of the affected area in accordance with a detection result of the movement of the affected area. According to the configuration described above, for example, observation of an affected area can be realized in a more preferable mode even in a situation where the affected area can move regardless of the presence or absence of an intervening procedure such as when an affected area that is a blood vessel or the like vibrates accompanying pulsation.

While a preferred embodiment of the present disclosure has been described in detail with reference to the accompanying drawings, the technical scope of the present disclosure is not limited thereto. It will be obvious to a person with ordinary skill in the art to which the present disclosure pertains that various modifications and changes may be made without departing from the technical ideas described in the appended claims and, as such, it is to be understood that such modifications and changes are to be naturally covered in the technical scope of the present disclosure.

In addition, the advantageous effects described in the present specification are merely descriptive or exemplary and not restrictive. In other words, the technique according to the present disclosure can produce, in addition to or in place of the advantageous effects described above, other advantageous effects that will obviously occur to those skilled in the art from the description of the present specification.

The following configurations are also covered in the technical scope of the present disclosure.

(1)

A medical observation system including:

an imaging portion configured to capture an image of an affected area; a detecting portion configured to extract a movement of a treatment tool held in a vicinity of the affected area based on images of the affected area having been sequentially captured by the imaging portion and to detect a movement of the affected area based on a result of the extraction; and

a control portion configured to control processing related to observation of the affected area in accordance with a detection result of the movement of the affected area.

(2)

The medical observation system according to (1), including an endoscope portion that includes a lens tube to be inserted into a body cavity of a patient, wherein

the imaging portion is configured to capture an image of the affected area having been acquired by the endoscope portion.

(3)

The medical observation system according to (1), including

a microscope portion configured to acquire an enlarged image of the affected area, wherein

the imaging portion is configured to capture the enlarged image having been acquired by the microscope portion.

(4)

A medical observation apparatus including;

a detecting portion configured to extract a movement of a treatment tool held in a vicinity of an affected area based on images of the affected area having been sequentially captured by an imaging portion and to detect a movement; of the affected area based on a result of the extraction; and

a control portion configured to control processing related to observation of the affected area in accordance with a detection result of the movement of the affected area.

(5)

The medical observation apparatus according to (4), wherein the control portion is configured to perform image processing with respect to a captured image of the affected area based on a detection result of a movement of the affected area.

(6)

The medical observation apparatus according to (5), wherein the control portion is configured to correct a blur of a captured image of the affected area based on a detection result of a movement of the affected area.

(7)

The medical observation apparatus according to (5) or (6), wherein the control portion is configured to perform control so that an image of the affected area before the image processing is performed and an image of the affected area after the image processing is performed are associated with each other and presented by an output portion.

(8)

The medical observation apparatus according to any one of (4) to (7), wherein the control portion is configured to cause an output portion to present display information based on a detection result of a movement of the affected area.

(9)

The medical observation apparatus according to (8), wherein the control portion is configured to control the display information to be presented by an output portion in accordance with a magnitude of a detected movement of the affected area.

(10)

The medical observation apparatus according to (9), wherein the control portion is configured to cause an output portion to present a warning as the display information when a magnitude of a detected movement of the affected area exceeds a threshold.

(11)

The medical observation apparatus according to (9) or (10), wherein the control portion is configured to cause an output portion to present information related to a procedure as the display information when a magnitude of a detected movement of the affected area is equal to or smaller than a threshold.

(12)

The medical observation apparatus according to any one of (4) to (11), wherein the control portion is configured to control conditions related to observation of the affected area based on a detection result of a movement of the affected area.

(13)

The medical observation apparatus according to (12), wherein the control portion is configured to control at least any of a shutter speed, an aperture, and a gain of the imaging portion in accordance with a magnitude of a detected movement of the affected area.

(14)

The medical observation apparatus according to (13), wherein the control portion is configured to execute at least any of control for increasing the shutter speed, control for opening the aperture, and control for increasing the gain when a magnitude of a detected movement of the affected area exceeds a threshold.

(15)

The medical observation apparatus according to any one of (4) to (14), wherein the detecting portion is configured to extract a movement of the treatment tool by detecting a light-emitting body held by the treatment tool from the sequentially captured images.

(16)

The medical observation apparatus according to any one of (4) to (14), wherein the detecting portion is configured to extract a movement of the treatment tool by detecting at least a portion having a predetermined feature among the treatment tool from the sequentially captured images.

(17)

The medical observation apparatus according to any one of (4) to (16), wherein the affected area is a blood vessel.

(18)

The medical observation apparatus according to (17), wherein the affected area is an aneurysm.

(19)

A drive method of a medical observation apparatus including the steps performed by a computer of;

extracting a movement of a treatment tool held in a vicinity of an affected area based on images of the affected area having been sequentially captured by an imaging portion and detecting a movement of the affected area based on a result of the extraction; and

controlling processing related to observation of the affected area in accordance with a detection result of the movement of the affected area.

(20)

A program causing a computer to perform the steps of:

extracting a movement of a treatment tool held in a vicinity of an affected area based on images of the affected area having been sequentially captured by an imaging portion and detecting a movement of the affected area based on a result of the extraction; and

controlling processing related to observation of the affected area in accordance with a detection result of the movement of the affected area.

### REFERENCE SIGNS LIST

2, 3 Medical observation system

165, 221, 225 Transmission cable

201 Control unit

203 imaging unit

205 Sensor driver

207 Input portion

209 Output portion

211 Imaging optical system

213 Branching optical system

215, 217 Imaging element

219 PGB laser

223 IR laser

227, 229 Vibration sensor

301 Control unit

303 imaging portion

305 Detecting portion

307 Output portion

309 Image analyzing portion

311 Vibration detecting portion

313 Imaging control portion

315 Image processing portion

317 Output control portion

1. A medical observation system comprising:
an imaging portion configured to capture an image of an affected area;
a detecting portion configured to extract a movement of a treatment tool held in a vicinity of the affected area based on images of the affected area having been sequentially captured by the imaging portion and to detect a movement of the affected area based on a result of the extraction; and
a control portion configured to control processing related to observation of the affected area in accordance with a detection result of the movement of the affected area.

2. The medical observation system according to claim 1, comprising an endoscope portion that includes a lens tube to be inserted into a body cavity of a patient, wherein
the imaging portion is configured to capture an image of the affected area having been acquired by the endoscope portion.

3. The medical observation system according to claim 1, comprising
a microscope portion configured to acquire an enlarged image of the affected area, wherein
the imaging portion is configured to capture the enlarged image having been acquired by the microscope portion.

4. A medical observation apparatus comprising:
a detecting portion configured to extract; a movement of a treatment tool held in vicinity of an affected area based on images of the affected area having been sequentially captured by an imaging portion and to detect a movement of the affected area based on a result of the extraction; and.
a control portion configured to control processing related to observation of the affected area in accordance with a detection result of the movement of the affected area.

5. The medical observation apparatus according to claim 4, wherein the control portion is configured to perform image processing with respect to a captured image of the affected area based on a detection result of a movement of the affected area.

6. The medical observation apparatus according to claim 5, wherein the control portion is configured to correct a blur of a captured image of the affected area based on a detection result of a movement of the affected area.

7. The medical observation apparatus according to claim 5, wherein the control portion is configured to perform control so that an image of the affected area before the image processing is performed and an image of the affected area after the image processing is performed are associated with each other and presented by an output portion.

8. The medical observation apparatus according to claim 4, wherein the control portion is configured to cause an output portion to present display information based on a detection result of a movement of the affected area.

9. The medical observation apparatus according to claim 8, wherein the control portion is configured to control the display information to be presented by an output portion in accordance with a magnitude of a detected movement of the affected area.

10. The medical observation apparatus according to claim 9, wherein the control portion is configured to cause an output portion to present a warning as the display information when a magnitude of a detected movement of the affected area exceeds a threshold.

11. The medical observation apparatus according to claim 9, wherein the control portion is configured to cause an output portion to present information related to a procedure as the display information when a magnitude of a detected movement of the affected area is equal to or smaller than a threshold.

12. The medical observation apparatus according to claim 4, wherein the control portion is configured to control conditions related to observation of the affected area based on a detection result of a movement of the affected area.

13. The medical observation apparatus according to claim 12, wherein the control portion is configured to control at least any of a shutter speed, an aperture, and a gain of the imaging portion in accordance with a magnitude of a detected movement of the affected area.

14. The medical observation apparatus according to claim 13, wherein the control portion is configured to execute at least any of control for increasing the shutter speed, control for opening the aperture, and control for increasing the gain when a magnitude of a detected movement of the affected area exceeds a threshold.

15. The medical observation apparatus according to claim 4, wherein the detecting portion is configured to extract a movement of the treatment tool by detecting a light-emitting body held by the treatment tool from the sequentially captured images.

16. The medical observation apparatus according to claim 4, wherein the detecting portion is configured to extract a movement of the treatment tool by detecting at least a portion having a predetermined feature among the treatment tool from the sequentially captured images.

17. The medical observation apparatus according to claim 4, wherein the affected area is a blood vessel.

18. The medical observation apparatus according to claim 17, wherein the affected area is an aneurysm.

19. A drive method of a medical observation apparatus comprising the steps performed by a computer of;
extracting a movement of a treatment tool held in a vicinity of an affected area based on images of the affected area having been sequentially captured by an imaging portion and detecting a movement of the affected area based on a result of the extraction; and
controlling processing related to observation of the affected area in accordance with a detection result of the movement of the affected area.
